(19) United States
(12) Patent Application Publication
(10) Pub. No.: US 2023/0414599 A1
(43) Pub. Date: Dec. 28, 2023

(54) NICOTINAMIDE, NICOTINAMIDE PRECURSORS AND NICOTINAMIDE METABOLITES AND COMPOSITIONS THEREOF FOR REDUCING THE TIME TO RESOLUTION OF SYMPTOMS IN PATIENTS WITH COVID-19 AND OTHER VIRAL INFECTIONS

(71) Applicant: CONARIS RESEARCH INSTITUTE AG, Kiel (DE)

(72) Inventors: Georg WÄTZIG, Kiel (DE); Stefan SCHREIBER, Kiel (DE)

(73) Assignee: CONARIS RESEARCH INSTITUTE AG, Kiel (DE)

(21) Appl. No.: 18/038,968

(22) PCT Filed: Nov. 26, 2021
(86) PCT No.: PCT/EP2021/083138

§ 371 Date: May 25, 2023

(30) Foreign Application Priority Data
Nov. 27, 2020 (EP) 20210479.0
Mar. 16, 2021 (EP) 21162960.5

(51) Int. Cl. A61K 31/455; A61P 31/14; A61K 9/00

(52) U.S. Cl. CPC A61K 31/455; A61P 31/14; A61K 9/0056

(57) ABSTRACT

The present invention relates to nicotinamide (niacinamide) or suitable precursors or metabolites thereof and compositions thereof for use for reducing the time to resolution of one or more symptoms in patients with COVID-19 or in patients with other viral infections.

### FIELD OF THE INVENTION

The present invention relates to nicotinamide (niacinamide) or suitable precursors or metabolites thereof and compositions thereof for use for reducing the time to alleviation or resolution of symptoms in patients with coronavirus disease 2019 (COVID-19) or in patients with other viral infections.

### BACKGROUND

The disease course following infection with the severe acute respiratory syndrome coronavirus type 2 (SARS-CoV-2) is highly variable, ranging from symptom-free infection to the development of severe acute respiratory syndrome with hospitalisation, eventual need for assisted ventilation and death. The vast majority of patients with SARS-CoV-2 infections that eventually develop COVID-19 comes down with symptoms which do not require hospital care. The leading trigger for hospital care are problems with gas exchange and ventilation, while general illness plays a secondary role. For unknown reasons, reduced organ functions after recovery from acute COVID-19 may persist for weeks to months after the acute phase of the infection in patients following both mild and severe disease courses. There are different terms for this syndrome, such as post-acute COVID-19 syndrome (PACS), post-acute sequelae of SARS-CoV-2 infection (PASC) or “long COVID”. Compared to common respiratory viral infections like influenza with similar symptoms (e.g. cough, fever or fatigue), Persistent Somatic Symptoms are much more common and severe in COVID-19 patients, particularly after hospitalization (Marshall 2020, Nature 585:339; Groff et al. 2021, JAMA Netw. Open 4:e2128568; Jiang et al. 2021, JACC Basic Transl. Sci. 6:796). Persistent Somatic Symptoms represent an umbrella term to describe subjectively distressing somatic complaints, irrespective of their aetiology, that are present on most days for at least several months after having recovered from an illness. Persistent Somatic Symptoms can be operationalised by repeated measures of patients' subjective somatic symptom severity and therefore include fatigue and other measures of suffering. In COVID-19, the persistence of symptoms or tissue damage beyond the acute phase of the disease is the rule rather than the exception: for example, 88% of participants in a study with COVID-19 patients still had visual lung damage 6 weeks after their discharge from hospital, and in 56% the damage persisted at 12 weeks (Marshall 2020, Nature 585:339). In another study, more than 70% of patients still suffered from shortness of breath one month after being discharged (Marshall 2020, Nature 585:339). In yet another study, 25% of patients had abnormal lung function after 3 months and 16% were still fatigued (Marshall 2020, Nature 585:339). A recent large meta-analysis showed that “more than half of COVID-19 survivors experienced PASC 6 months after recovery” (Groff et al. 2021, JAMA Netw. Open 4:e2128568). For related coronaviruses like SARS-CoV-1, symptom persistence has also been described and has been shown to last for years (Marshall 2020, Nature 585:339). This persistence of symptoms has been suggested to be connected to the durability of antibodies and immune responses in ˜100 day longitudinal follow-up (Chen at al. 2020, Cell 183:1). In any case, the persistence of symptoms is not restricted to hospitalized patients with severe COVID-19 as described above, but occurs in a similar fashion in patients with mild disease. For example, persistent fatigue following COVID-19 was independent from disease severity in a cohort of 128 patients from Ireland, of which 52.3% reported persistent fatigue at a median of 10 weeks after initial COVID-19 symptoms (Townsend et al. 2020, PloS ONE 15:e0240784).

In a follow-up study of a post-acute outpatient service for patients discharged from a hospital in Rome (Italy) after recovery from COVID-19, 143 patients were included and assessed at a mean of 60.3 days after onset of the first COVID-19 symptoms (Carfi et al. 2020, JAMA 324:604). At this time, 87.4% of patients reported persistence of at least 1 COVID-19-related symptom, with 32.2% reporting 1 or 2 symptoms and 55.2% reporting 3 or more symptoms. The most frequently remaining symptoms were fatigue (53.1%), dyspnea (43.3%) and joint pain (27.3%) (Carfi et al. 2020, JAMA 324:604).

The COVID-19 Response Team of the United States Centers for Disease Control and Prevention (CDC) conducted a large study comprising telephone interviews by CDC personnel with a site-specific random sample of adults that had first been tested SARS-CoV-2-positive at an outpatient visit at one of 14 academic health care systems in 13 states (Tenforde et al. 2020, MMWR 69:993). Interviews were conducted 14-21 days after this test date in the period from April to June 2020. Among 292 respondents, 94% (274) had one or more COVID-19 symptoms at the time of their initial test and were further analysed. The median age of symptomatic respondents was 42.5 years, 52% were female and 53% of respondents with available respective data (141 of 264) had one or more chronic medical conditions. The median interval from test to interview date was 16 days. Among 270 of 274 interviewed symptomatic patients with available respective data, 95 (35%) had not returned to their usual state of health at the time of the interview. The proportion of such patients increased with age and the number of chronic conditions: from 26% (18-34 years) to 47% (>50) and from 28% (no or one chronic condition) to 57% (three or more), respectively. Overall, the most commonly reported symptoms were fatigue (71%), cough and headache (both 61%), with cough and fatigue being the least likely to have resolved 14-21 days after testing positive (43% and 35%, respectively). Importantly, in addition to the 35% of patients (95 of 270) that had not returned to their usual state of health at that time, those reporting to have returned to their usual state of health were not all symptom-free: 34% (59 of 175) still suffered from one or more of the 17 queried COVID-19-related symptoms. Taken together, the study of the CDC demonstrated that 154 (95+59) of 270 patients (57%) were not symptom-free 14-21 days after their initial positive testing for SARS-CoV-2. Moreover, symptoms that commenced after the date of testing were not even recorded in the survey (Tenforde et al. 2020, MMWR 69:993).

Between March and June 2020, a descriptive clinical follow-up of 150 COVID-19 patients with non-critical COVID-19 was performed at days 7, 30 and 60 after the patients had tested SARS-CoV-2-positive at the University Hospital of Tours in France (Carvalho-Schneider et al. 2021, Clin. Microbiol. Infect. 27:258). It was found that 68% (103 of 150) patients retained at least one symptom of their COVID-19 disease at day 30 and, even more strikingly, 66% (86 of 130) at day 60. The main persistent symptoms at day 30/day 60 were asthenia (49.3%/40%), dyspnea (36.7%/30%) and anosmia/ageusia (28%/23%). The authors concluded that “up to 2 months after symptom onset, two thirds of adults with non-critical COVID-19 had complaints, mainly anosmia/ageusia, dyspnea or asthenia” (Carvalho-Schneider et al. 2021, Clin. Microbiol. Infect. 27:258).

Complementing data were provided from a follow-up cohort of 384 patients with COVID-19, in which, at a median of 54 days post discharge from hospital, 53% reported persistent breathlessness, 34% cough and 69% fatigue (Mandal et al. 2021, Thorax 76:396).

In a cohort of 669 ambulatory patients from Geneva (Switzerland), at least 32% of the patients reported one or more symptoms at 30 to 45 days (mean, 43 days) from diagnosis. Fatigue, dyspnea, and loss of taste or smell were the main persistent symptoms (Nehme et al. 2021, Ann. Intern. Med. 174:723).

In addition to the well-known respiratory symptoms, gastrointestinal manifestations of COVID-19 receive increasing attention, as emerging epidemiological data suggest an association between gastrointestinal injury induced by SARS-CoV-2 infection and the clinical features, prognosis, and disease severity of COVID-19 (Mitsuyama et al. 2020, J. Clin. Med. 9:3630). Typical gastrointestinal symptoms include loss of appetite, nausea, vomiting, diarrhea, and abdominal pain, which may be caused and accompanied by small and large bowel wall thickening, fluid-filled colon, pneumatosis intestinalis, pneumoperitoneum, intussusception, and ascites [reviewed by Lui et al. 2021, Abdom. Radiol. (NY) 46:1249]. In a hospitalised cohort of patients with COVID-19, 45.6% showed acute mucosal injuries and 33.3% presented with ischaemic-like colitis (Vanella et al. 2021, BMC Open Gastroenterol. 8:e000578). Moreover, the disturbance of the composition of the intestinal microbiota (dysbiosis) has been shown to reflect disease severity and dysfunctional immune responses in patients with COVID-19 (Yeoh et al. 2021, Gut 70:698). In American patients with risk factors such as overweight, obesity or other comorbidities, gastrointestinal symptoms have been observed in the majority (61.3%) of patients, with loss of appetite (34.8%), diarrhea (33.7%) and nausea (26.4%) being the most common symptoms (Redd et al. 2020, Gastroenterology 159:765). Patients with gastrointestinal symptoms were also significantly more prone to other COVID-19 symptoms, such as fatigue (65.1% compared to 45.5% in patients without gastrointestinal symptoms) or myalgia (49.2% vs. 22%) (Redd et al. 2020, Gastroenterology 159:765).

Symptomatic suffering in COVID-19 can be extensive and most patients report their distress to be majorly caused by somatic symptom states (Repišti et al. 2020, Global Psychiatry 3:201). So far, there is no medication or other intervention that can substantially shorten the duration of symptoms in the large proportion of patients with mild to moderate COVID-19 in domestic quarantine or primary care. In contrast, anti-inflammatory therapies like prednisone are associated with worsened courses if given during early disease (Brenner et al. 2020, Gastroenterology 159:481), which is different from dexamethasone given during acute respiratory distress syndrome (The RECOVERY Collaborative Group 2021, N. Engl. J. Med. 384:693).

Therefore, there is a large unmet need for active substances and compositions with no or minimal side effects that can reduce the time to alleviation or resolution of COVID-19 symptoms. Given the pathophysiology of COVID-19, where viral replication leads to cellular damage and hence a chain of pathophysiologic events including endothelial cell death and coagulation activation, such agents most likely require more than anti-inflammatory or antiviral activities.

The term vitamin B3 comprises nicotinic acid and nicotinamide. In addition to being a precursor of the pivotal and ubiquitous coenzymes nicotinamide adenine dinucleotide (NAD) and its phosphorylated derivative nicotinamide adenine dinucleotide phosphate (NADP), nicotinamide is also involved in energy homoeostasis signalling pathways in intestinal epithelial cells and in maintaining the secretion of antimicrobial peptides from these cells (Hashimoto et al. 2012, Nature 487:477). With regard to maintaining the health and functionality of intestinal epithelial cells and the gut microbiota, nicotinamide has an efficacy similar to that of its precursor, the essential amino acid tryptophan (Hashimoto et al. 2012, Nature 487:477). Accordingly, sufficient amounts of tryptophan or nicotinamide are not only particularly important in fast replicating cells like epithelial cells to fuel energy metabolism, but supplementation of nicotinamide also protects from dysregulation of the intestinal microbiota and intestinal inflammation, particularly when the nicotinamide is topically delivered by appropriate formulations or compositions to the lower small intestine and large intestine where the microbiota are located (Hashimoto et al. 2012, Nature 487:477; Waetzig & Seegert 2013, PCT/EP2013/062363; Bettenworth et al. 2014, Mol. Nutr. Food Res. 58:1474; Watzig & Seegert 2015, PCT/EP2014/077637; Watzig & Seegert 2015, PCT/EP2014/077646). Moreover, supplementation of NAD precursors like nicotinamide has been suggested to be beneficial in fighting coronavirus infections (Heer et al. 2020, J. Biol. Chem. 295:17986). However, using nicotinamide is not the one and obvious choice, as nicotinamide riboside has been suggested to be more efficacious in raising NAD levels (Bogan-Brown et al. 2021, J. Diet. Suppl., DOI: 10.1080/19390211.2021.1881686). Nicotinamide is authorised for use in food [Regulation (EC) No 1925/2006, amended by Commission Regulation (EC) No 1170/2009], in food supplements (Directive 2002/46/EC) as well as in infant and follow-on formula, baby food and food for particular nutritional uses (Regulation (EU) No 609/2013). Nicotinamide is mainly marketed in the form of dietary supplements, although there are also nicotinamide prescription drugs for treating vitamin B3 deficiency. Nicotinamide has an excellent safety profile, resulting in a high Tolerable Upper Intake Level (UL) or lifelong Acceptable Daily Intake (ADI) of 12.5 mg/kg/d or 900 mg/d as defined by the European Food Safety Authority (EFSA 2002, SCF/CS/NUT/UPPLEV/39; EFSA 2014, EFSA J. 12:3759).

There is evidence for various virus types that nicotinamide can reduce viral replication and support the body's defence mechanisms, e.g., in the case of vaccinia virus (Child et a. 1988, Virus Res. 9:119), human immunodeficiency virus (Murray 2003, Clin. Infect. Dis. 36:453), enteroviruses (Moell et al. 2009, J. Med. Virol. 81:1082) or hepatitis B virus (Li et a. 2016, Arch. Virol. 161:621). Due to its putatively favourable risk/benefit ratio, sufficient supplementation of nicotinamide in COVID-19 patients has been recommended because of its antiviral activity and protective effects against lung injury and bacterial lung infections (Shi eta. 2020, Cell Death Differ. 27:1451; Gharote 2020, Ind. J. Med. Sci. 72:25; Zhang et a. 2020, J. Med. Virol. 92:479; Mehmel et a. 2020, Nutrients 12:1616; Shakoor et a. 2021, Maturitas 144:108). Replenishment of NAD levels by NAM supplementation is considered to restore antiviral innate immune functions and rebalance the maladaptive, hyperinflammatory immune response to SARS-CoV-2 infection (Mehmel et al. 2020, Nutrients 12:1616).

The intestinal uptake of tryptophan, the precursor of nicotinamide, depends on the presence of angiotensin converting enzyme-2 (ACE2) on the intestinal epithelium (Hashimoto et al. 2012, Nature 487:477). ACE2 is required for SARS-CoV-2 to enter human body cells and is expressed at much higher levels in intestinal than in respiratory epithelia (Mitsuyama et al. 2020, J. Clin. Med. 9:3630). SARS-CoV-2 infection inevitably reduces the cell surface expression of ACE2, which leads to malabsorption of tryptophan, gut dysbiosis and intestinal inflammatory symptoms (Hashimoto et al. 2012, Nature 487:477; Mitsuyama et a. 2020, J. Clin. Med. 9:3630; Yeoh et a. 2021, Gut 70:698; Vanella et a. 2021, BMC Open Gastroenterol. 8:e000578). This could be compensated by administration of nicotinamide, the uptake of which does not depend on ACE2 and which is equally effective in maintaining intestinal health and a healthy gut microbiota (Hashimoto et a. 2012, Nature 487:477).

However, there are proposals for many other different active substances for use in alleviation of SARS-CoV-2-associated symptoms.

Other viral infections also lead to shifts in tryptophan metabolism. For example, in influenza, metabolization of tryptophan to kynurenine—a precursor of nicotinamide—by indoleamine 2,3-dioxygenase-1 (IDO1) is increased, potentially by a direct reaction to viral infection (van der Sluijs et al. 2006, J. Infect. Dis. 193:214; Lin et a. 2020, Acta Otolaryngol. 140:149). Enhanced IDO1 activity and tryptophan removal correlate closely with IL-6-linked biomarkers of inflammation (i.e., neopterin) and to prognosis of patients (Pizzini et a. 2019, Influenza Other Respir. Viruses 13:603; Pett et a. 2018, Open Forum Infect Dis. 5:ofx228). It appears that interferon release plays a particular role in IDO1 induction that consecutively channels tryptophan to kynurenine (Gaelings et al. 2017, FEBS J. 284:222). During the recent outbreak of H1 N1 influenza, the degradation of tryptophan was among the most informative markers predicting development of acute respiratory distress syndrome (Ferrarini et al. 2017, Electrophoresis 38:2341). An interesting cross-connection has been proposed between gut and lung immune-microbiome interactions in which D-tryptophan generated by probiotic bacteria appears to influence allergic airway disease via changes in the gut microbiome (Kepert et al. 2017, J. Allergy Clin. Immunol. 139:1525). In severe cases of influenza, like in chronic inflammation, increased tryptophan degradation and thus increased kynurenine levels are observed, and inhibition of tryptophan degradation has positive effects in animal models (Boergeling & Ludwig 2017, FEBS J. 284:218; Pizzini et al. 2019, Influenza Other Respir. Viruses 13:603).

Taken together, the state of the art suggests that nicotinamide might act on immune mechanisms as a natural, but non-specific antiviral active substance in nutritional or pharmaceutical formulations administered to patients with COVID-19. There is evidence that nicotinamide riboside may be more suitable than nicotinamide. Tryptophan and the blockade of interleukin-6 may synergize in their antiviral activity by regulation of interferon gamma (Belladonna & Orabona 2020, Front. Pharmacol. 11:959). Besides strong anti-inflammatory agents like dexamethasone or the interleukin-6 blocker tocilizumab with its limited and strongly disease stage-dependent efficacy (Hermine et al. 2021, JAMA Intern. Med. 181:32; Gupta et al. 2021, JAMA Intern. Med. 181:41; Stone et al. 2020, N. Engl. J. Med. 383:2333; Gordon et al. 2021, N. Engl. J. Med. 384:1491; Rosas et al. 2021, N. Engl. J. Med. 384:1503), the only approved targeted antiviral treatment of COVID-19 is remdesivir, which targets the coronavirus RNA polymerase. Remdesivir has been shown to significantly shorten the median time to recovery from 15 to 10 days in severely ill hospitalized adults (Beigel et al. 2020, N. Engl. J. Med. 383:1813; Goldman et al. 2020, N. Engl. J. Med. 383:1827). Its low efficacy can be improved by combining it with the Janus kinase inhibitor baricitinib (Kalil et al. 2021, N. Engl. J. Med. 384:795) but has no real impact in clinical routine. Recently, encouraging key results from an interim analysis of the MOVe-OUT trial investigating the antiviral molnupiravir were released to the press by Merck and Ridgeback Therapeutics, suggesting that molnupiravir may reduce the risk of hospitalization or death by approximately 50% compared to placebo in patients with mild or moderate COVID-19. Even more encouraging interim results of the EPIC-HR trial were recently released to the press by Pfizer, describing a 89% reduction in hospitalization and death when non-hospitalised COVID-19 patients at high risk of progressing to severe illness received a combination of the SARS-CoV-2-3CL protease inhibitor PF-07321332 and a low dose of ritonavir (a combination named PAXLOVID™) compared to placebo. In both trials and in many other currently ongoing trials, the enrolled patients were and are required to have at least one characteristic or underlying medical condition associated with an increased risk of developing severe illness from COVID-19.

However, the definition of recovery applied in most of these trials and also in other intervention trials with biologics is according to the WHO definition of progression to severe COVID-19 and means by far not the absence of general COVID-19-related symptoms, i.e. health, but focused on clinical status score categories assessed on 7-point (Goldman et al. 2020, N. Engl. J. Med. 383:1827) or 8-point (Beigel et al. 2020, N. Engl. J. Med. 383:1813) ordinal scales. For example, the 7-point ordinal scale used by Goldman et al. 2020 (N. Engl. J. Med. 383:1827) consisted of the following categories: 1, death; 2, hospitalized, receiving invasive mechanical ventilation or extracorporeal membrane oxygenation; 3, hospitalized, receiving noninvasive ventilation or high-flow oxygen devices; 4, hospitalized, requiring low-flow supplemental oxygen; 5, hospitalized, not requiring supplemental oxygen but receiving ongoing medical care (related or not related to Covid-19); 6, hospitalized, requiring neither supplemental oxygen nor ongoing medical care (other than that specified in the protocol for remdesivir administration); and 7, not hospitalized. The current standard is the World Health Organization (WHO) COVID-19 Ordinal Scale for Clinical Improvement (available from: https://wwwwhoint/publications/i/item/covid-19-therapeutic-trial-synopsis). Such measures reflects the scope of development and the assigned capabilities of antiviral drugs, which by reducing viral load will have an impact on deterioration of disease and is neither intended to nor capable of providing symptomatic relief. Remdesivir alone offered no significant reduction in the time to recovery in moderately ill hospitalized COVID-19 patients (Spinner et al. 2020, JAMA 324:1048) and its usefulness has been questioned because of remdesivir's inefficacy in the WHO SOLIDARITY trial and its risk-benefit ratio (Hsu 2020, BMJ 371:m4457). The combination of remdesivir and baricitinib provided a faster time to recovery of 10 days versus 15 days with placebo, but only in patients receiving non-invasive ventilation of high-flow oxygen, and with the limitation that recovery was defined not as resolution of symptoms, but included conditions up to hospitalisation without oxygen requirement (Kalil et al. 2021, N. Engl. J. Med. 384:795). Monoclonal antibodies directed against SARS-CoV-2 like bamlanivimab alone or in combination with etesevimab (Chen et al. 2021, N. Engl. J. Med. 384:229; NCT04427501) as well as a combination of casirivimab and imdevimab (Weinreich et al. 2021, N. Engl. J. Med. 384:238) show some promise in limiting early COVID-19, which is reflected by emergency use authorizations from the United States Food and Drug Administration. However, their efficacy is very limited and these therapeutics may face risk/benefit and benefit/cost issues. Therefore, apart from—possibly soon—molnupiravir and PAXLOVID™, there is presently neither an available, cost-efficient therapy ameliorating the symptoms of the general suffering from COVID-19 in an effective way nor one that reliably reduces the percentage of patients moving from hospitalization into a severe course of the disease.

Analogous antivirals like favipiravir, which targets the influenza RNA polymerase (Udwadia et al. 2021, Int. J. Infect. Dis. 103:62), have likewise shown promise but no definitively proven efficacy against COVID-19. In general, the untargeted administration of different antivirals under the desperate conditions of the first COVID-19 wave in China in early 2020 suggested that early treatment with any antiviral may quicken virus clearance and reduce the likelihood of a severe disease course of COVID-19 (Yu et al. 2020, J. Med. Virol. 92:2675). Antivirals are expected to increase viral clearance as an objective endpoint and to reduce disease scores, e.g., like those described above for COVID-19 (Beigel et al. 2020, N. Engl. J. Med. 383:1813), by supporting the immune system in fighting the infection. With regard to SARS-CoV-2, the long established strategies employed against influenza viruses and pandemics are the closest available prior art (Ison et al. 2010, J. Infect. Dis. 201:1654; Mifsud et al. 2019, Antiviral Res. 169:1045; Uyeki et al. 2019, Clin. Infect. Dis. 68:e1). For antivirals targeting influenza, criteria like time to recovery, time to alleviation or resolution of symptoms, time to resumption of usual activities or time to return to prior level of care are used as endpoints in clinical trials (Ison et al. 2010, J. Infect. Dis. 201:1654; Mifsud et al. 2019, Antiviral Res. 169:1045; Uyeki et al. 2019, Clin. Infect. Dis. 68:e1). Usually, antivirals are not used to ameliorate or eliminate the symptoms of the disease, for which other, primarily symptom-modifying drug classes are more appropriate (e.g., analgesics to relieve pain, antipyretics to ameliorate fever or cough suppressants to mitigate dry cough), even though clinical experience shows that these drugs usually do not these are frequently not very effective in viral infections.

Compared to influenza and other respiratory viral infections (see above), the time to recovery and particularly the time to resolution of symptoms is much longer in COVID-19 patients, regardless of the severity of their disease course (Marshall 2020, Nature 585:339; Chen at al. 2020, Cell 183:1; Carfi et al. 2020, JAMA 324:604; Tenforde et al. 2020, MMWR 69:993; Carvalho-Schneider et al. 2021, Clin. Microbiol. Infect. 27:258). Therefore, it was not to be expected by a person skilled in the art that treatment with a non-specific antiviral agent like nicotinamide would shorten the time to complete resolution of symptoms in COVID-19 patients with mild to moderate disease, but—if at all—rather ameliorate the disease course in those patients that would otherwise be prone to a severe course and require hospitalization with oxygen or mechanical ventilation.

However, a treatment that shortens the time of resolution of symptoms in COVID-19 and other virus-related diseases is desperately needed to reduce the suffering from acute disease, to avoid prolongation of functional deficits into long-term symptoms and, consequently, to reduce the number of sick days for patients, especially for those with COVID-19, also in those with mild to moderate disease, which is not only a health problem, but also a large socioeconomic problem.

Therefore, it was an object of the present invention to provide means for reducing the time to resolution (not only alleviation) of symptoms in patients with virus-related diseases, especially with COVID-19. Preferably, the resolution of symptoms should be reached within a short time period for a high percentage of patients. In addition to that, it was an object of the present invention to provide means for a post-exposure prophylaxis for individuals that were exposed to the pathogens causing the respective diseases.

According to the invention, this object is solved by a composition comprising an active substance selected from nicotinamide; nicotinic acid; nicotinic acid esters; tryptophan; a tryptophan dipeptide; nicotinamide adenine dinucleotide (NAD); nicotinamide adenine dinucleotide phosphate (NADP); an intermediate in the biosynthesis of NAD or NADP selected from the group consisting of N-formylkynurenine, L-kynurenine, 3-hydroxy-L-kynurenine, 3-hydroxyanthranilate, 2-amino-3-carboxymuconate semialdehyde, quinolinate, nicotinic acid mononucleotide (beta-nicotinate 0-ribonucleotide), and nicotinic acid adenine dinucleotide; nicotinamide riboside; nicotinamide mononucleotide; 1-methylnicotinamide/N-methylnicotinamide; or a combination thereof, for use for reducing the time to resolution of one or more, preferably all symptoms related to a disease selected from the group consisting of coronavirus disease 2019 (COVID-19), severe acute respiratory syndrome (SARS), middle-east respiratory syndrome (MERS), influenza, acquired immunodeficiency syndrome (AIDS), hepatitis type A, hepatitis type B, hepatitis type C, hepatitis type 0, hepatitis type E, enterovirus infection and vaccinia virus infection, wherein the composition is formulated to partly or completely release the active substance, preferably nicotinamide, for topical supplementation or efficacy in the lower small intestine and/or the colon.

Preferably, the composition according to the invention is formulated for oral administration.

The human body is a metaorganism incorporating human cells and a multitude of microbial species, particularly in the intestinal microbiota. The sum of all metabolic pathways available from all parts of this metaorganism has to be viewed as a whole, and can be used by the human body either directly or indirectly. Tryptophan and nicotinic acid cannot be synthesised de novo by human cells, but can be taken up from diverse sources from the gut. The active substances listed above can be synthesised as intermediates in synthesis pathways leading from these precursors to NAD or NADP. Therefore, these substances are considered functionally equivalent to the particularly safe and well-characterised NAD(P) precursor nicotinamide. For example, in human nutrition science, the term “niacin equivalent” already indicates the interchangeability of the precursor tryptophan and its products nicotinic acid and nicotinamide: approximately 60 mg of tryptophan yields 1 mg of niacin defined as 1 mg niacin equivalent (EFSA Scientific Opinion on dietary reference values for niacin; EFSA Journal 2014; 12:3759). For reasons of conciseness, the description and exemplification of the present invention focuses on nicotinamide without objective restriction to this compound. However, nicotinamide is the most preferred active substance.

Preferably, at least one symptom in case of COVID-19 is selected from the group listed in Table 2 of Example 5.

More preferably, at least one symptom in case of COVID-19 is selected from the group consisting of the reduced ability to perform normal activities, reduced physical performance, fatigue, cough with or without sputum, shortness of breath, impaired sense of smell and/or taste, sore throat, joint pain, and/or chest pain.

In the sense of this specification, symptoms may be self-assessed by patients (e.g., by interviews and/or questionnaires and/or mobile apps, particularly regarding multidimensional parameters of general well-being like the ability to perform normal activities) and/or by objective examinations and tests (e.g., by physical examinations to measure, e.g., shortness of breath, by electronic device monitoring of activities and/or physical parameters and/or, e.g., by smell tests to detect and/or quantify an impaired sense of smell). In case of doubt, the assessment of the respective symptoms is made via self-assessment.

Moreover, the present invention also comprises the analogous use of the active substances in other viral infections with at least some symptoms overlapping with COVID-19 symptoms, particularly (prolonged) fatigue, for the prevention of symptoms, preferably Persistent Somatic Symptoms (particularly fatigue) of viral infections, including but not limited to SARS-CoV-1, SARS-CoV-2, middle-east respiratory syndrome coronavirus (MERS-CoV); influenza; human immunodeficiency virus; hepatitis virus type A, B, C, D or E; enterovirus; or vaccinia virus.

The person skilled in art understands that in the sense of the present invention, a “resolution of symptoms” only can occur if such symptoms were present.

As used herein, “resolution of symptoms” means that symptoms that where present were completely eliminated at least according to the impression of the patient.

Those symptoms are for
SARS-CoV-2 infection: dyspnoea/shortness of breath; cough; whistling/wheezing; pneumonia; fatigue; reduced physical performance; reduced ability to perform normal activities; impaired sense of taste and/or smell; headache; rhinitis/rhinorrhoea; chest pain; fever; chills; sore throat/pharyngitis/pharyngalgia; hoarseness; sputum production; diarrhoea; joint pain/arthralgia; limb pain/melalgia; muscle pain/myalgia; abdominal pain; anorexia/loss of appetite/lower food intake; nausea; vomiting; disturbed consciousness; dizziness; confusion; disturbed sleep; skin rash; conjunctivitis; hair loss;
SARS-CoV-1 infection: fatigue; fever; dry cough; dyspnoea/shortness of breath;
MERS-CoV: infection: fatigue; fever; dry cough; dyspnoea/shortness of breath; diarrhea; nausea; vomiting;
influenza virus infection: fatigue; fever; chills; cough; sore throat; rhinorrhoea; nasal congestion;
myalgia; body pain; headache; vomiting; diarrhea;
human immunodeficiency virus infection: fatigue; recurring fever; profuse night sweats; weight loss;
prolonged swelling of lymph nodes in the armpits, groin or neck; diarrhoea; sores of the mouth, anus or genitals; pneumonia;
hepatitis virus type A infection: fatigue/malaise; loss of appetite; diarrhea; nausea; abdominal discomfort; dark-coloured urine; jaundice;
hepatitis virus type B infection: fatigue; fever; loss of appetite; nausea; vomiting; abdominal pain; dark-coloured urine; clay-coloured bowel movements;
hepatitis virus type C infection: fatigue; sore muscles; joint pain; fever; nausea; loss of appetite;
stomach pain; itchy skin; dark urine; jaundice;
hepatitis virus type D infection: fatigue/malaise; loss of appetite; abdominal pain; jaundice; dark-coloured urine; clay-coloured bowel movements;
hepatitis virus type E infection: fatigue; abdominal pain; loss of appetite; nausea; vomiting; fever;
jaundice; dark-coloured urine; clay-coloured bowel movements;
enterovirus infection: fever; rhinorrhoea; sneezing; cough; rash; mouth blisters; body aches; myalgia;
vaccinia virus infection: skin lesions; fever; swollen lymph nodes; fatigue/malaise; body aches.

According to the invention, the problem stated above is preferably solved by the use of nicotinamide in a supplementation or treatment regimen or a composition comprising nicotinamide, as defined in the claims and/or described in more detail herein. The use of suitable precursors or metabolites of nicotinamide, alone or in combination, together with or instead of nicotinamide, is also in the scope of the present invention. For reasons of conciseness, this is not repeated in all instances, but nicotinamide is used as a preferred example. In preferred embodiments, the composition comprising nicotinamide is formulated to partly or completely release the nicotinamide in the lower small intestine and/or the colon to beneficially and topically influence the intestinal mucosa and the intestinal microbiota as described in the following patent families: Waetzig & Seegert 2013, PCT/EP2013/062363; Watzig & Seegert 2015, PCT/EP2014/077637; Watzig & Seegert 2015, PCT/EP2014/077646; Schwarz et al. 2017, PCT/EP2017/058733. For example, nicotinamide is formulated to be released selectively, e.g., for at least partially topical efficacy, in the lower small intestine and/or colon, where the intestinal microbiota are located. Accordingly, compositions are provided which preferably contain nicotinamide which acts in a beneficial manner on the disease course and particularly the time to alleviation or resolution of symptoms of preferably COVID-19.

The invention also includes means for the prevention of symptoms of related to COVID-19 or other viral infections by providing a composition as definded above for use as a post-exposure prophylaxis to prevent the onset of symptoms related to a disease selected from the group consisting of COVID-19, SARS, MERS, influenza, AIDS, hepatitis type A, hepatitis type B, hepatitis type C, hepatitis type 0, hepatitis type E, enterovirus infection and vaccinia virus infection in patients that were tested positive for the respective pathogen.

### BRIEF DESCRIPTION OF THE DRAWINGS

FIG. 1 shows a schematic summary of the beneficial effects of nicotinamide and compositions comprising nicotinamide for the supplementation and treatment of patients with COVID-19.

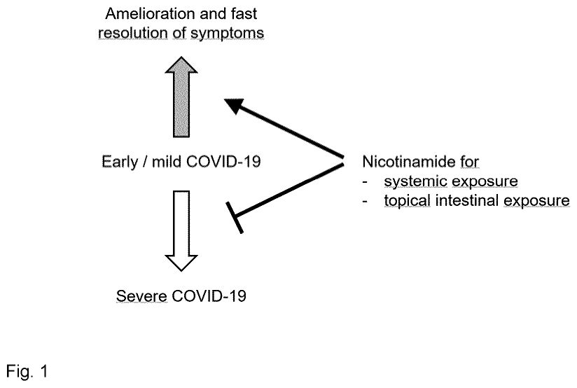

FIG. 2 shows the correlation of the ratio of tryptophan and C-reactive protein with the severity of the disease course in two cohorts of patients hospitalized for COVID-19. CRP, C-reactive protein; TRP, tryptophan.

FIG. 3 shows a degradation map of tryptophan and its metabolites in patients hospitalized for COVID-19 grouped for patients remaining on regular wards (box plots on the left in each panel) and patients requiring admission to the intensive care unit (ICU—box plots on the right in each panel). 3-HK, 3-hydroxykynurenine; 3-HAA, 3-hydroxyanthranilic acid; CRP, C-reactive protein; IAA, indole-3-acetic acid; KYN, kynurenine; NAM, nicotinamide; QUI, quinolinic acid; TRP, tryptophan.

FIG. 4 shows a graphical representation of the differences between a pilot group of COVID-19 patients supplementing nicotinamide and those supplementing silica (placebo) [each n=8; pilot phase of the first part of the COVit trial (COVit-1)] with regard to the time to resolution of symptoms.

FIG. 5 shows a graphical representation of the percentage of COVID-19 patients [n=56 enrolled in the first part of the COVit trial (COVit-1); n=28 each supplementing nicotinamide or silica (placebo)] with certain remaining symptoms.

FIG. 6 shows Kaplan-Meier graphs for completely symptom-free COVID-19 patients under dietary supplementation with nicotinamide (NAM) or silica (placebo) in the first part of the COVit trial (COVit-1); n=28 per group.

FIG. 7 shows box plots with medians and interquartile ranges (IQR) representing the time to complete resolution of symptoms in patients after 4 weeks of dietary supplementation with nicotinamide (NAM) or silica (placebo) in the first part of the COVit trial (COVit-1); n=28 per group.

### DETAILED DESCRIPTION

While it can be anticipated that chronic inflammation is associated with degradation of tryptophan (Nikolaus et al. 2017, Gastroenterology 153:1504) and reduced tryptophan levels have been suggested in COVID-19 (Essa et al. 2020, Int. J. Tryptophan Res. 13:1), it was surprisingly observed that a low ratio of tryptophan to the inflammation biomarker C-reactive protein (CRP) at the time of admission to the hospital was strongly correlated to the severity of the course of COVID-19 and, particularly, that the unexpectedly strong decrease in tryptophan serum levels did not result in increased availability of nicotinamide as one of the key metabolites, which would have been expected by the person skilled in the art (Example 1).

Moreover, in the pilot phase of the first part of the COVit trial (COVit-1; Example 2), it was surprisingly found that supplementation of 1,000 mg nicotinamide per day for 28 days reduced the number of subjects still suffering from at least one COVID-19 symptom at or before day 21 after first testing positive for SARS-CoV-2 to a proportion of 50% (4 of 8) compared to a placebo control group receiving silica, in which 87.5% of patients (7 of 8) still presented with one or more COVID-19 symptoms (Example 2). One patient in the placebo group, but no patient in the nicotinamide group, required hospitalisation before week 2. This effect appeared very strong in the recruited patient population, but was also clearly evident when the symptom-free proportion of patients was compared to results obtained with a comparable European population from France in which 68% (103 of 150) patients retained at least one symptom of their COVID-19 disease at Day 30 and, even more strikingly, 66% (86 of 130) at Day 60 (Carvalho-Schneider et al. 2021, Clin. Microbiol. Infect. 27:258). When this long-term outcome is compared to the results from week 6 in the pilot trial (Example 2), it is clear that the patient population recruited for the COVit-1 trial and particularly the silica (control) group had no unusually unfavourable disease course or prognosis, but was well comparable to the published cohorts.

The signal was confirmed with an amplitude of approximately 20 percentage points (35.7% symptom-free after 2 weeks with nicotinamide, 14.3% with placebo/silica), when the patient population was expanded to n=28 per group in the context of the COVit-1 trial (Example 5). Surprisingly, female patients selectively benefited from conventional nicotinamide supplementation in this setting (Example 5).

The positive signal from COVit-1 justified a larger confirmatory trial part (COVit-2), for which interim data (n=402 patients) are available (Example 6). The key difference to COVit-1 is the placebo-controlled use of 2 different nicotinamide tablets: one conventional 500-mg immediate-release nicotinamide tablet (the same as used in COVit-1) and one novel 500-mg controlled-ileocolonic-release nicotinamide (CICR-NAM) tablet, which ensures prolonged and continuous intestinal exposure to nicotinamide. This concept has been developed for the improvement of the intestinal microbiota, particularly in the treatment of inflammatory bowel diseases, and was intended to ameliorate gastrointestinal symptoms of COVID-19 (see Background). The use of a combination of immediate-release and controlled-ileocolonic-release nicotinamide surprisingly led to significant improvements in the ability to perform normal activities, physical performance and fatigue in patients with at least one characteristic or underlying medical condition associated with an increased risk of developing severe illness from COVID-19 as well as in patients with key symptoms of COVID-19 (Example 6). More surprisingly, and in contrast to the use of conventional nicotinamide only (as in COVit-1; Examples 2 and 5), the combination of immediate- and controlled-ileocolonic-release nicotinamide (i) was particularly efficacious in improving these key parameters of general well-being and (ii) was beneficial in both males and females (Example 6).

An especially preferred aspect of the present invention is the surprisingly successful pragmatic dosing regimen of Examples 2, 5 and 6: While the recommended intake for normal vitamin B3 supply is less than 20 mg/day, which is far below the ADI of 12.5 mg/kg/d or 900 mg/d (EFSA 2002, SCF/CS/NUT/UPPLEV/39; EFSA 2014, EFSA J. 12:3759), the effective supplementation in the study described in Examples 2, 5 and 6 was 1.000 mg/day administered once daily with breakfast in the morning. In contrast, a typical supplementation regimen aiming at keeping exposure permanently high and trough levels between doses as high as possible would have used repeated dosing of several smaller doses (e.g., 3×300 mg with meals in the morning, at noon and in the evening).

Therefore, the core of the present invention is the use of preferably nicotinamide in a supplementation or treatment regimen to reduce the time to resolution of one or more symptoms in patients with COVID-19, or a composition comprising nicotinamide for oral administration for this use, as defined in the claims and/or described in more detail herein. As shown in Example 5, the rapid recovery of the sense of taste and smell in COVID-19 patients under nicotinamide supplementation is a preferred effect of the invention. In particular, improvements in the ability to perform normal activities, physical performance and fatigue in patients with at least one characteristic or underlying medical condition associated with an increased risk of developing severe illness from COVID-19 as well as in patients with key symptoms of COVID-19 are preferred effects of the invention. The use of preferably nicotinamide as a post-exposure prophylaxis after positive testing for SARS-CoV-2 and before the onset of disease symptoms is also within the scope of the present invention. Moreover, the present invention also comprises the analogous use of nicotinamide in other viral infections with at least some symptoms overlapping with COVID-19 symptoms, particularly the reduced ability to perform normal activities, reduced physical performance and/or fatigue, for the prevention or amelioration of Persistent Somatic Symptoms (particularly the reduced ability to perform normal activities, reduced physical performance and/or fatigue) of viral infections including but not limited to SARS-CoV-1, SARS-CoV-2, middle-east respiratory syndrome coronavirus (MERS-CoV); influenza; human immunodeficiency virus; hepatitis virus type A, B, C, D or E; enterovirus; or vaccinia virus.

In addition to the preferred active substance nicotinamide, suitable precursors or metabolites of nicotinamide can be used in the invention as active substances. For example, compounds that convert into nicotinamide (e.g., by hydrolysis or metabolism) in the human or animal body are suitable, such as nicotinic acid or nicotinic acid esters. In addition, intermediates in the biosynthesis of nicotinamide adenine dinucleotide (NAD) or NAD phosphate (NADP) starting from tryptophan, such as N-formylkynurenine, L-kynurenine, 3-hydroxy-L-kynurenine, 3-hydroxyanthranilate, 2-amino-3-carboxymuconate semialdehyde, quinolinate, nicotinic acid mononucleotide (beta-nicotinate 0-ribonucleotide), and nicotinic acid adenine dinucleotide, can be used. Further examples include NAD, NADP, nicotinamide riboside, nicotinamide mononucleotide or the nicotinamide metabolite 1-methylnicotinamide/N-methylnicotinamide. Dipeptidic tryptophan as an equivalent for nicotinamide in case of compromised ACE2 cell surface expression in the intestine (Hashimoto et al. 2012, Nature 487:477) is also in the scope of the present invention. The use of these suitable precursors or metabolites of nicotinamide, alone or in combination, together with or instead of nicotinamide, is also in the scope of the present invention. For reasons of conciseness, this is not repeated in all instances, but nicotinamide is used as a preferred example.

In general, the uses disclosed in this invention may be medical uses or non-medical uses. Medical use in the sense of the present application preferably means that the composition for use according to the invention is a medicament, authorised by the respective competent regulatory authority of the respective country where the use takes place, and wherein all other uses are non-medical uses.

It is preferred that the composition according to the invention is formulated for oral administration to partly or completely release nicotinamide for topical supplementation or efficacy in the lower small intestine and/or the colon to beneficially and topically influence the intestinal mucosa and the intestinal microbiota as described in the following patent families: Waetzig & Seegert 2013, PCT/EP2013/062363; Watzig & Seegert 2015, PCT/EP2014/077637; Watzig & Seegert 2015, PCT/EP2014/077646; Schwarz et al. 2017, PCT/EP2017/058733. Preferably, the composition according to the invention is formulated to be released selectively, more preferably for at least partially delayed release of nicotinamide for topical supplementation or efficacy, in the lower small intestine and/or colon, where the intestinal microbiota are located. Preferably, the composition according to the invention is formulated to start releasing nicotinamide in the second half of the jejunum. Alternatively preferred, the composition according to the invention is formulated to start releasing in the terminal ileum and/or colon. In a further preferred embodiment, the release of nicotinamide in both delayed and non-delayed dosage forms is prolonged by an extended-release and/or controlled-release formulation to achieve higher trough levels and a more constant systemic exposure.

In the present invention, the terms “formulation” or “composition” or “supplementation” or “treatment”, and in particular the term “composition”, have a broad meaning of a pharmaceutically and/or nutritionally and/or physiologically acceptable formulation, composition and/or mode of administration of nicotinamide, which includes, but is not limited to, medicaments (pharmaceutical formulations), nutraceuticals, foods for special medical purposes, dietary supplements, food ingredients and/or foods. The nature of the composition may vary, e.g., depending on the ingredients and excipients, the dose of nicotinamide, the formulation type and other factors. Preferred are dietary supplements, food for special medical purposes, nutraceuticals and medicaments.

The composition according to the invention preferably is formulated for at least partly delayed release of the active substance, preferably of nicotinamide, for topical supplementation or efficacy in the lower small intestine and/or the colon.

The composition according to the invention preferably comprises one or more nicotinamide formulations for immediate release and/or extended release and/or sustained release delivering nicotinamide mainly systemically to the circulation together with one or more nicotinamide formulations for delayed release and/or delayed-controlled release delivering nicotinamide mainly topically to the lower small intestine and/or colon. For definitions of delayed and delayed-controlled release, see below.

The composition according to the invention preferably contains a combination of two formulation variants of nicotinamide in a specific ratio by weight in the range of from 1:1 to 1:1000, preferably from 1:3 to 1:300, more preferably from 1:10 to 1:100.

A combination may be present in the same or separate dosage forms, which may be administered simultaneously or sequentially. The composition may be suitable for oral administration with immediate and/or extended and/or sustained release to achieve systemic exposure to nicotinamide by delivering it to the circulation. Preferably, the composition according to the invention may be suitable for delayed release and/or delayed-controlled release of nicotinamide for specific local or topical efficacy in the lower small intestine and/or colon.

As used herein, the terms “preferred” or “preferably” refer to embodiments that may have certain benefits under certain circumstances, but other embodiments may also be preferred under the same or other circumstances. The recitation of one or more preferred embodiments does not imply exclusion of other useful embodiments from the scope of the invention. Terms like “comprises” and variations thereof do not have a limiting meaning in the description and claims. Citation of certain sections of documents from the literature does not imply that the rest of such documents is not relevant or not incorporated by reference. The recitations of numerical ranges by one or two endpoints includes all numbers subsumed within that range (e.g., “1 to 10” includes 1, 2.4, 4.576, etc., and “lower than 1” includes all numbers smaller than 1). For any method disclosed or cited herein that includes discrete steps, the steps may be conducted in any feasible order, and any combination of two or more steps may be conducted simultaneously. Any example or list of examples should not be interpreted as a restriction of any kind or as an exclusive list.

As used herein, the term “supplementation” refers to dietary supplementation of nicotinamide in patients, preferably those with COVID-19. As used herein, the terms “treatment,” “treat,” and “treating” refer to reversing, alleviating, or inhibiting the progress of the diseases mentioned herein, preferably of COVID-19, or one or more symptoms thereof by administration of nicotinamide, as described herein. In some embodiments, supplementation or treatment may be administered after one or more symptoms have developed. In other embodiments, supplementation or treatment may be administered in the absence of symptoms. For example, supplementation or treatment may be administered to a SARS-CoV-2-infected individual prior to the onset of symptoms. Supplementation or treatment may also be continued after symptoms have resolved, for example to prevent or delay their recurrence.

As used herein, the “lower small intestine” is the second half of the small intestine comprising the second half of the jejunum and the ileum. As used herein, the “terminal ileum” is the second half of the ileum.

As used herein, the term “topical efficacy” refers to a topical effect, in the pharmacodynamic sense, and thus refers to a local, rather than systemic, target for a dietary supplementation or medication. Accordingly, “local supplementation or efficacy” means a local supplementation or therapy with nicotinamide released specifically or selectively at a location where, for example, the dietary supplement or food ingredient or medication shall deliver its direct effect and nicotinamide enters the circulation to a lower degree than from conventional formulations with immediate and/or extended and/or sustained release, e.g., thereby causing only a reduced or low systemic action compared to conventional formulations. In this regard, the topical efficacy of the present invention is also contrasted with enteral (in the digestive tract) and intravascular/intravenous (injected into the circulatory system) administrations. In comparison to compositions aiming at high systemic availability and/or exposure, the at least partially topical efficacy of compositions may also be characterized by longer latency times until systemic levels of nicotinamide increase. Such latency times for topical release can be correlated with intestinal transit times known in the art (see, e.g., Davis et al. 1986, Gut 27:886; Evans et al. 1988, Gut 29:1035; Kararli 1995, Biopharm. Drug Dispos. 16:351; Sutton 2004, Adv. Drug Deliv. Rev. 56:1383). For example, after a variable time for gastric emptying (depending on the dosage form and feeding status and ranging from <15 minutes to more than 10 hours), small intestinal transit times are rather constant with 1-6 (usually 2-4) hours across formulations and studies (Davis et al. 1986, Gut 27:886). Thus, in case of doubt, the latency time in a fasted patient would usually be at least 2 hours for formulations with topical efficacy, at which time a formulation reaches the lower small intestine and systemic levels of nicotinamide may start to rise strongly. In the context of the present invention, topical efficacy can also be expressed in terms of a reduction of the plasma peak levels of at least 20%, 30%, 40%, 50%, 60%, 70%, 80%, 90% or even 95% or more relative to the same amount of nicotinamide administered in immediate-release formulations (e.g., nicotinamide in a capsule that dissolves in the stomach) in the same way and under the same conditions. As baseline values may differ strongly also within the same person, it is preferred to refer the peak levels to the respective baseline level immediately before the administration. Preferably, average plasma levels of a suitable cohort of persons are used for this definition of topical efficacy rather than the respective levels of single persons, which can yield highly divergent results (Schwarz et al. 2017, PCT/EP2017/058733). Topical efficacy is achieved in particular by the composition according to the invention as described herein. In combination formulations comprising nicotinamide formulations for immediate release and/or extended release and/or sustained release delivering nicotinamide mainly systemically to the circulation together with one or more nicotinamide formulations for delayed release and/or delayed-controlled release delivering nicotinamide mainly topically to the lower small intestine and/or colon, the above reduction of peak levels applies only to the delayed-release or delayed-controlled-release formulation, respectively.

It has previously been demonstrated that nicotinamide has a surprising anti-inflammatory effect by influencing the intestinal microbiota (the entirety of all microorganisms in the intestines, in particular the bacteria), which are mainly located in the lower small intestine and in the colon (Waetzig & Seegert 2013, PCT/EP2013/062363; Watzig & Seegert 2015, PCT/EP2014/077637; Watzig & Seegert 2015, PCT/EP2014/077646). The mechanism behind this surprising effect has been shown to involve nicotinamide-induced changes in the secretion pattern of antimicrobial peptides in the intestines, which supports the maintenance and/or regeneration of the normal, healthy intestinal microbiota (Hashimoto et a. 2012, Nature 487:477).

Therefore, as used herein, “beneficially influencing the intestinal microbiota” refers to causing a change in the intestinal microbiota that has a beneficial impact on health, especially on one or more of the diseases and conditions described herein, and/or to maintaining the healthy intestinal microbiota in preventive settings. For example, beneficial impacts may be associated with reducing the number of pathogenic bacteria, reducing the ratio of pathogenic bacteria to beneficial bacteria, increasing the diversity of the microbiota, increasing the amount of beneficial bacteria, partly or completely reverting pathological changes in the enterotype of the microbiota (e.g., enterotypes associated with Bacteroides, Prevotella and Ruminococcus) and/or maintaining the healthy endogenous microbiota.

Thus, preferred according to the invention is a composition according to the invention for oral administration with at least partially delayed and/or delayed-controlled release of the active substance (preferably nicotinamide) for specific local supplementation or efficacy in the lower small intestine and/or the colon. More preferred, the composition is formulated for oral administration with at least partially delayed release of the active substance for specific local supplementation or efficacy in the lower small intestine and/or the colon. In another more preferred variant, the composition is formulated for oral administration with at least partially delayed-controlled release of nicotinamide for specific local supplementation or efficacy in the lower small intestine and/or the colon.

Preferably, nicotinamide is used in a dietary and/or pharmacological formulation that protects at least part of the nicotinamide from being absorbed by the body, e.g., from being absorbed into the circulatory system, in the upper small intestine and rather effects an at least partially topical release (e.g., delayed release and/or delayed-controlled release) into the lower small intestine and/or colon.

In particular, nicotinamide and the formulations and compositions described herein are thus suitable for being used in medicaments, nutraceuticals, foods for special medical purposes, dietary supplements, food ingredients and/or foods with at least partially topical release (e.g., delayed release and/or delayed-controlled release) to also enable direct nicotinamide supplementation or treatment of the lower small intestine and/or the colon, e.g., in the context of COVID-19 and its gastrointestinal symptoms, and/or a prolonged resorption period due to the continuous intestinal exposure.

Nicotinamide and the formulations and compositions described herein are equally usable in SARS-CoV-2 infections in both human and other mammals, in particular in domestic and useful animals. Examples of such animals are dogs, cats, minks, horses, camels, pigs or cows without objective restriction.

Nicotinamide may be used in any form available on the market in suitable nutritional or pharmaceutical quality, e.g., provided by general manufacturers and vendors like DSM, Lonza or Merck.

The present invention also comprises combination preparations and/or compositions of nicotinamide, such as a variable dose combination or a fixed dose combination of immediate-release, sustained-release, extended-release, delayed-release and/or delayed-controlled-release nicotinamide. The different release kinetics of such formulations may be used to tailor the extent, duration and kinetics of systemic exposure and topical intestinal exposure to nicotinamide. The combinations described herein may be present in the same or separate dosage forms, which may be administered simultaneously or sequentially. The composition and dosage of such combinations is known to a person skilled in the art.

As used herein, the term “variable dose combination” refers to a combination of two or more formulation variants of nicotinamide in medicaments, nutraceuticals, foods for special medical purposes, dietary supplements, food ingredients and/or foods, whereby each formulation variant of nicotinamide is applied in the form of a separate composition, e.g., two single dosage forms. The separate compositions may be administered simultaneously, sequentially or on separate occasions by an administration regimen.

For example, a composition of immediate-release nicotinamide (to be quickly absorbed after entering the stomach) in any suitable dosage thereof may be administered together, consecutively or subsequently, with a separate composition of delayed-release nicotinamide (to be partly or completely protected from absorption until reaching the lowed small intestine) in any suitable dosage thereof. Thus, variable dosages of two or more different formulations of nicotinamide may be combined. These variable dose combinations may use conventionally available compositions of medicaments, nutraceuticals, foods for special medical purposes, dietary supplements, food ingredients and/or foods or may be also achieved by customized polypharmacy via compounding. Depending on the presence and severity of gastrointestinal symptoms of a patient and/or expected benefits from a prolonged resorption period due to the continuous intestinal exposure, immediate-release, sustained-release or extended-release for systemic delivery and delayed-release or delayed-controlled-release for topical intestinal delivery may be administered in different proportions and dosages.

In contrast to a variable dose combination, a “fixed-dose combination” as used herein is a combination which is a formulation including two or more formulation variants of nicotinamide either combined in a single dosage form, which is manufactured and distributed in certain respective fixed doses, or in a combination of two or more separate dosage forms representing formulation variants of which a fixed number or amount is to be supplemented or administered according to label. A fixed-dose combination mostly refers to a mass-produced product having a predetermined combination and respective dosages.

The total dosage of the active substance (preferably nicotinamide) used according to the invention can be in the range of from 1 to 5000 mg, which may be administered as an individual dosage or as multiple dosages and/or a once, twice or more often daily dosage. The preferred total dosage of the active substance according to the invention is in the range of from 10 to 4000 mg, more preferably in the range of from 100 to 3000 mg.

As a non-limiting example, a high dose formulation can comprise up to 5000 mg of the active substance. For example, but not limited to, a high dose formulation can comprise a total of the active substance in the range of 1000-5000 mg, preferably in the range of 1000-4000 mg, more preferably in the range of 1000-3000 mg, e.g., 2000 mg.

As a non-limiting example, a low dose formulation can comprise up to 1000 mg, and preferably in a range of 1-1000 mg of the active substance, more preferably in a range of 100-1000 mg, of the active substance.

As a non-limiting example, a standard dose formulation can comprise up to 3000 mg, and preferably in a range of 250-2500 mg, more preferably in a range of 500-2000 mg, of the active substance.

A non-limiting particular example of a fixed-dose high dose formulation comprises a combination of 1000 mg immediate-release nicotinamide and 1000 mg delayed-release and/or delayed-controlled-release nicotinamide.

A non-limiting particular example of a fixed-dose standard dose formulation comprises a combination of 750 mg immediate-release nicotinamide and 750 mg delayed-release and/or delayed-controlled-release nicotinamide.

A non-limiting particular example of a fixed-dose low dose formulation comprises a combination of 400 mg or 500 mg immediate-release nicotinamide and 400 or 500 mg delayed-release and/or delayed-controlled-release nicotinamide.

Such compositions of the invention may, for example, preferably be administered as tablets, pellets or granulates, preferably microgranulates, if suitable in a capsule, sachet or stick pack, and preferably in a sachet or stick pack.

It is preferred that the active substance (preferably nicotinamide) is formulated in the form of tablets, granules, microgranules or pellets. These tablets, granules, microgranules or pellets can be used for single dosage forms or for variable dose combinations or fixed dose combinations. If different formulation variants of the active substance in the form of tablets, granules, microgranules or pellets are used as described herein, these may be used in the form of any single dietary or pharmaceutical composition, as well as a variable dose combination or a fixed dose combination. Granules, microgranules or pellets may be compressed into tablets, or filled into capsules, sachets or stick packs, or used as such, as appropriate.

In order to produce orally administered formulations of the active substance (e.g., tablets, dragees, capsules, sachets, etc.) for at least partial release in the lower small intestine and/or in the colon, it is advantageous to use delayed modes of release. In contrast to conventional (in some cases also delayed, but systemically delivering) modes of release for optimum supplementation of certain embodiments of the present invention, e.g., immediate-release, extended-release and/or sustained-release nicotinamide formulations, such delayed or and/or delayed-controlled modes of release (at least) partially or (even) substantially avoid an absorption in the stomach and in the upper portions of the small intestine.

For oral administration, particular dosage forms that at least partially control and/or delay the release of the active substance due to special galenics are particularly suitable. Such dosage forms may be simple tablets and also coated tablets, e.g., film tablets or dragees. The tablets are usually oblong, round or biconvex. Particular oblong tablet forms, which allow the tablet to be separated, can be preferred. In addition, minitablets, granules, spheroids, pellets or microcapsules are possible (e.g., Liang & Dingari 2017, PCT/US2017/028063; Schwarz et al. 2017, PCT/EP2017/058733), which are filled into capsules, sachets or stick packs, where appropriate. In order to deliver nicotinamide in part in a systemically acting formulation (e.g., immediate release) and in a formulation acting largely topically on the lower small intestine and/or colon (e.g., delayed and/or delayed-controlled release), combinations of different formulations in separate dosage forms and/or multilayer dosage forms can be used to first release part of the active substance in the stomach and upper small intestine and release the other part from, e.g., a quickly disintegrating core (delayed release) or a matrix core (delayed-controlled release) with or without pH-dependent or microbial-dependent release. Another example are erosion-based release technologies exemplified by the OralogiK™ product portfolio (BOO Pharma).

The term “delayed release” relates preferably to a formulation or component thereof that releases, or delivers, the active substance after a period of delay, e.g., degradation of a film coating or other coating due to the pH, chemical, enzymatic and/or microbial environment that is preferably present in the lower small intestine and/or colon. In certain embodiments, the delay is sufficient for at least a portion of the active substance in a formulation to be released in the lower small intestine and/or colon.

The term “delayed-controlled release” refers preferably to a formulation or component thereof that releases, or delivers, the active substance over a prolonged period of time (time-dependent release) and/or under certain physiological conditions, e.g., degradation of a coating or matrix due to the pH, chemical, enzymatic and/or microbial environment that is preferably present in the lower small intestine and/or colon. In certain embodiments, the period of time or the release according to physiological conditions is sufficient for at least a portion of the nicotinamide in a formulation to be released in the lower small intestine and/or colon.

The retardation and/or delayed release and/or delayed-controlled release is advantageously achieved, e.g., by coatings which are resistant to gastric juice and dissolve depending on the pH, by using different carrier matrix components (e.g., different grades of hydroxypropyl cellulose, hydroxypropyl methylcellulose, microcrystalline cellulose, sodium carboxymethylcellulose, starch, modified starch, pregelatinized starch, gelatin, polyvinylpyrrolidone) or combinations thereof, by means of other matrix and/or multi-matrix (MMX) technologies, or a combination of these techniques. Examples include film coatings which contain acrylic and/or methacrylate polymers in various mixtures for delayed release. Additional examples include biodegradable polymers like natural or chemically modified polymers and polymer-drug conjugates, coatings and/or matrix agents for microbiota-dependent release (reviewed, e.g., by Rajpurohit et al. 2010, Indian J. Pharm. Sci. 72:689). For example, the active substance can be contained in a matrix comprising components as described above, which is coated with a material that provides the delayed release of the active substance. The active substance according to the invention can be administered in, e.g., tablets, minitablets, granules, spheroids, pellets, microcapsules or large-volume capsules (e.g., gelatin or hydroxypropyl methylcellulose capsules), which are coated by means of known methods. Suitable coating agents are water-insoluble waxes, such as carnauba wax, and/or polymers, such as poly(meth)acrylates, e.g., the entire poly(meth)acrylate product portfolios with the trade names Eudraguard® and Eudragit® provided by from Evonik Industries, in particular Eudraguard® protect, Eudraguard® control, Eudraguard® biotic, Eudraguard® natural, Eudragit® L 30 D-55 (an aqueous dispersion of anionic polymers with methacrylic acid as a functional group), Eudragit® L 100-55 (which contains an anionic copolymer based on methacrylic acid and ethyl acrylate), Eudragit® L 100 or L 12,5 or S 100 or S 12,5 (anionic copolymers based on methacrylic acid and methyl methacrylate), combinations of Eudragit® S and L compounds, or Eudragit® FS 30 D (an aqueous dispersion of an anionic copolymer based on methyl acrylate, methyl methacrylate and methacrylic acid), and/or water-insoluble celluloses (e.g., methyl cellulose or ethyl cellulose). Where appropriate, water-soluble polymers (e.g., polyvinylpyrrolidone), water-soluble celluloses (e.g., hydroxypropylmethyl cellulose or hydroxypropyl cellulose), emulsifiers and stabilisers (e.g., polysorbate 80), polyethylene glycol (PEG), lactose or mannitol can also be contained in the coating material.

In preferred embodiments, formulations for immediate release and/or extended release and/or sustained release are equipped with taste-masking technologies comprising, alone or in combination, e.g.,
organoleptic methods, such as adding combinations of sweeteners (e.g., sucralose, aspartame, acesulfame potassium, glycerrhizin, cyclamate, lactose, mannitol, saccharin, or sucrose), sugars, flavours (e.g., mint, peppermint, menthol, wild cherry, walnut, chocolate, passion fruit or citrus flavours like lemon or orange), bitterness-blocking agents (e.g., adenosine monophosphate, dihydrochalone, sodium chloride, sodium acetate, sodium gluconate, lipoproteins [e.g., composed of phosphatidic acid and β-lactoglobulin] or phospholipids [e.g., phosphatidic acid, phosphatidylinositol or soy lecithin]), effervescent agents (e.g., generators of carbon dioxide), taste-modifiers, buffers and/or other excipients (e.g., zinc sulfate, maltodextrins [e.g., pea maltodextrin] or polyols) to the composition or a coating thereof;
single- or multi-layer coatings and/or matrix technologies comprising, alone or in combination, e.g., hydrophobic or hydrophilic polymers (e.g., methacrylic acid and methacrylic ester copolymers like Eudragit® E, E-100, RL 300, RS 300, L30D-55 or NE 30D; Eudraguard® protect, natural or control; ethylcellulose; hydroxypropylmethylcellulose; hydroxypropylcellulose; cellulose acetate; croscarmellose; polyvinyl alcohol; polyvinylpyrrolidone (e.g., PVP-K30 or Kollicoat); polyvinyl acetate; shellac; guar gum), lipids (e.g., glyceryl palmitostearate, glyceryl monostearate or glycerol behenate), talc, detergents (e.g., sodium lauryl sulfate or polysorbates like polysorbate 80), sugars and/or sweeteners (see above);
matrix granulation (e.g., with gelling or lipid polymers);
solid dispersions using melting, solvent or melting-solvent methods and carriers like povidone, polyethylene glycols of various molecular weights, hydroxypropylmethylcellulose, urea, mannitol or ethylcellulose; spray-drying (e.g., with ethylcellulose, hydroxypropylmethylcellulose; hydroxypropylcellulose or acrylate polymers); hot-melt extrusion (e.g., with ethylcellulose, hydroxypropylmethylcellulose; hydroxypropylcellulose or acrylate polymers), preferably followed by milling or micronizing of the extrudates to obtain taste-masked granules or particles, which are preferably subsequently incorporated into a suitable dosage form;
microencapsulation using the coating agents listed above (e.g. Wurster fluid bed coating [e.g., with croscarmellose, Eudragit]);
barrier membranes;
inclusion complexation (e.g., by cyclodextrins, tannic acid, Eudragit® polymers like Eudragit S-100 or chitosan);
ion exchange resins, such as copolymers of styrene, acrylic acid or methacrylic acid with divinylbenzene;
adsorption (e.g. using silicates, silica gel or bentonite).

Further non-limiting examples especially for the formulation of dietary supplements, but also food ingredients, according to the present invention have been described using maltodextrin-pectin microcapsules and shellac-coated granulates (Berg et al. 2012, J. Food Eng. 108:158; Schwarz et al. 2017, PCT/EP2017/058733; Theismann et al. 2019, Int. J. Pharm. 564:472).

The composition, e.g., a formulation of medicaments, nutraceuticals, foods for special medical purposes, dietary supplements, food ingredients and/or foods, can also contain further excipient substances, such as binders (e.g., methylcellulose, carboxymethylcellulose sodium, hydroxypropyl cellulose, hydroxypropyl methylcellulose/hypromellose, hydroxyethyl cellulose, dextrin, maltodextrin, copovidone and/or sodium alginate), fillers (e.g., anhydrous lactose, lactose monohydrate, starch, pregelatinized starch, powdered cellulose, calcium carbonate, magnesium carbonate, anhydrous dibasic calcium phosphate, dibasic calcium phosphate dihydrate, anhydrous calcium sulfate, calcium sulfate dihydrate, tribasic calcium phosphate, sucrose, fructose, anhydrous glucose/dextrose, glucose/dextrose monohydrate, sorbitol, mannitol, maltitol, isomalt and/or xylitol), glidants, lubricants and flow regulating agents. The nicotinamide according to the invention can be formulated, where appropriate, together with further active substances and with excipients conventional in dietary or pharmaceutical compositions, e.g., talcum, gum arabic, lactose, starch, magnesium stearate, cocoa butter, aqueous and non-aqueous carriers, lipid components of animal or vegetable origin, paraffin derivatives, glycols (in particular polyethylene glycol), various plasticizers, dispersants, emulsifiers and/or preservatives.

A further aspect of the invention described herein is the efficient use of the described medicaments, nutraceuticals, foods for special medical purposes, dietary supplements, food ingredients and/or foods on the basis of blood and/or urine and/or stool and/or genetic and/or microbiological and/or other biomarkers or data and specific needs of the individuals to be treated. In particular, serum levels of tryptophan and its metabolites can be used to direct supplementation or therapeutic decisions (Example 1). Evidence-based personalized medicine including analyses and data of the disease course, the virus (preferably SARS-CoV-2) variant and/or the genetic background (e.g., genes coding for cell surface receptors, transporter proteins, metabolism enzymes or signal transduction proteins, which interact with the virus, the immune response to the virus, nicotinamide and/or its metabolites and/or its downstream effectors) can contribute information and improvements with respect to the type of use, the mode of application, the time(s) of use, the dose and/or the dosage regimen of the medicaments, nutraceuticals, foods for special medical purposes, dietary supplements, food ingredients and/or foods described herein. Individuals who may benefit from this personalised treatment include those with disease-specific or non-specific changes in blood and/or plasma and/or serum lipids and/or other biomarkers. This applies analogously to analyses of the intestinal microbiota, particularly when a stool sample indicates a change in the microbiota. The present invention thus also comprises the use of suitable test methods to identify individuals particularly susceptible to the medicaments, nutraceuticals, foods for special medical purposes, dietary supplements, food ingredients and/or foods according to the invention and/or to adapt the use of these as well as concomitant supplementation and/or medication to the individual circumstances. This also comprises expressly the use of different formulation variants or compositions comprising the active substance or combinations thereof in different modes of administration depending on the biomarkers of the individual to be supplemented or treated. For these purposes, it is possible to use laboratory tests and/or suitable test kits and also measuring methods, devices and/or kits to be employed by a physician, user and/or patient, e.g., to analyze suitable parameters in the blood, urine or other body fluids or in stool samples. In particular, the present invention also relates to using these biomarkers to support patient or subject selection for the supplementation or treatment described herein, to personalise and adapt the compositions and/or supplementations and/or treatments described herein, and/or to determine end points and efficacy benchmarks for the compositions and/or supplementations and/or treatments described herein.

As can be seen from the examples and from above, it is preferred according to the invention that the composition according to the invention is formulated for use for administration once daily. Surprisingly, this regimen of administration showed superior effects. The best effects were gained when the composition according to the invention was administered with breakfast in the morning. For dosing once daily, a nicotinamide content of 1 to 5000 mg per finished dosage form, preferably 50 to 4000 mg and more preferably 100 to 3000 mg is preferred.

As described above and can be seen from the examples, the composition according to the invention was very effective in the resolution of symptoms of the diseases described herein, especially COVID-19. Accordingly, a composition according to the invention is preferred for use in the resolution of one or more symptoms and/or for use in the improvement of the ability to perform normal activities, physical performance and/or fatigue within four weeks, preferably within three weeks, more preferably within two weeks in patients tested positive for a viral disease as described above. Preferably, this effect is achieved at least for 10%, preferably 15%, more preferably 20%, even more preferably 25% and most preferably 30% of those patients. It is alternatively or additionally preferred that the effect is achieved in a similar percentage of male and female patients, wherein similar means+/−25%, preferably +/−10%.

It should further be mentioned that according to the invention, it is most preferred that the disease is COVID-19.

The composition according to the invention is preferably used for administration to patients with at least one characteristic or underlying medical condition associated with an increased risk of developing severe illness from the viral infection, wherein preferably the characteristic or underlying medical condition is selected from the group consisting of a body mass index of at least 30.0 (obesity), type 2 diabetes, status of current smoker, and status of former smoker.

Alternatively or in addition to that, a composition according to the invention is preferred for a use wherein treatment and/or supplementation is administered to patients with at least one characteristic symptom of COVID-19, preferably selected from the group consisting of cough, fever and taste loss.

### EXEMPLIFICATION

There are variable possibilities to advantageously develop, and develop further, the teaching of the present invention. For this purpose, reference is made to the examples below which describe the invention in a representative way.

### Example 1: Tryptophan Metabolism in Patients with COVID-19

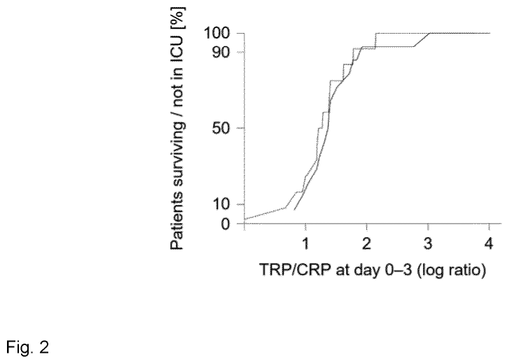

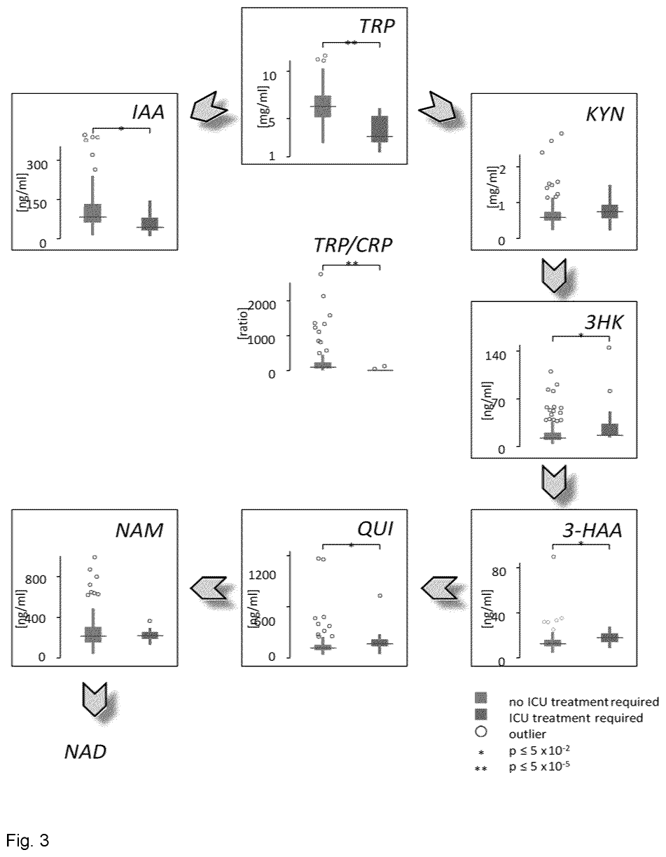

In two cohorts of patients hospitalized for COVID-19 from different hospitals, serum levels of tryptophan, a precursor of nicotinamide, were decreased to an unusual extent, and a low ratio of tryptophan to the inflammation biomarker CRP at the time of admission to the hospital was strongly correlated to the severity of the course of COVID-19 (i.e., the likelihood of being admitted to intensive care with assisted ventilation and/or the likelihood of death) (FIG. 2). Subsequent analyses of tryptophan degradation products demonstrated that surprisingly, nicotinamide as a key metabolite of tryptophan was not increased, as could be expected from the levels of the direct metabolic intermediates between tryptophan and nicotinamide, but nicotinamide was obviously removed from availability (FIG. 3).

### Example 2: Results from the Pilot Phase of the COVit-1 Dietary Intervention Trial in COVID-19 Patients with Mild to Moderate Disease

In the pilot phase of a monocentric, randomized, double-blind, prospective, placebo-like-controlled dietary intervention trial (“COVit-1”, DRKS-ID: DRKS00021214) conducted at the Department of Internal Medicine I of the University Medical Center Schleswig-Holstein, Campus Kiel (Germany), outpatients with early symptomatic COVID-19 in domestic quarantine were recruited from April to June 2020 and randomized to orally self-administer either 1,000 mg immediate-release nicotinamide (2×500 mg tablets of a marketed dietary supplement formulation) or a capsule with 245 mg silica as a placebo-like control once daily with breakfast in the morning, for 4 weeks (28 days). Inclusion criteria were a laboratory-confirmed SARS-CoV-2 infection, COVID-19 symptoms in the respiratory or gastrointestinal tract and an age of ≥18 years. There were no exclusion criteria. According to the known properties of nicotinamide (see Background), the primary endpoint of the trial was the frequency of hospital admission for at least 24 h of continuous oxygen therapy, and secondary endpoints included frequencies of machine ventilation, intensive care, death as well as time to resolution of symptoms.

The entire trial was conducted remotely. Patients registered online and were contacted by the study team to check their eligibility and to supply the written patient information and informed consent forms. After informed consent, patients were called for baseline data acquisition (week 0) and subsequently at week 2, week 4 and week 6. The queried baseline information included personal and demographic data, smoking status, comorbidities, concomitant administration of dietary supplements or medicaments as well as COVID-19 symptoms. At weeks 2, 4 and 6, regular intake of the trial supplements and concomitant supplements and medicaments was queried as well as the current COVID-19 symptoms and disease course.

After 16 outpatients (for baseline characteristics, trial group assignments and symptoms, see Tables 1, 2 and 3, respectively), data quality control tests were performed and inadvertently revealed that the last secondary endpoint which was considered the least likely to be met, i.e., time to resolution of symptoms, showed a surprising difference between the nicotinamide and the silica (control) group.

TABLE 1
Patient characteristics

| | Total | Male | Female |
|---|---|---|---|
| n | 16 | 5 | 11 |
| Age [years] | 41.5 ± 11.68 | 43.6 ± 11.97 | 40.54 ± 12.01 |
| Height [cm] | 173.88 ± 8.81 | 182.00 ± 5.29 | 170.18 ± 7.56 |
| Weight [kg] | 75.75 ± 11.61 | 86.20 ± 6.94 | 71.00 ± 10.17 |
| Smoking status | | | |
| Smoker (current) | 1 | 0 | 1 |
| Smoker | 3 | 1 | 2 |
| (previous) | | | |
| Non-smoker | 12 | 4 | 8 |

TABLE 2
Assignment of trial intervention (randomized, double blind)

| | Total | Male | Female |
|---|---|---|---|
| Nicotinamide | 8 | 2 | 6 |
| Silica | 8 | 3 | 5 |

TABLE 3
Symptoms at baseline

| Symptoms at baseline sorted by frequency | Total population (n = 16) |
|---|---|
| Fatigue | 14 |
| Cough | 14 |
| Headache | 13 |
| Anorexia/loss of appetite | 12 |
| Reduced physical performance | 11 |
| Fever (total) | 11 |
| Rhinorrhoea | 11 |
| Fever >38° C. | 10 |
| Impaired sense of taste | 10 |
| Sore throat/pharyngalgia | 9 |
| Impaired sense of smell | 8 |
| Fever <38° C. | 7 |
| Dyspnoea/shortness of breath | 6 |
| Myalgia | 5 |
| Chest pain | 5 |
| Sputum production | 4 |
| Diarrhoea | 4 |
| Arthralgia | 3 |
| Vomiting | 2 |

It was surprisingly found that supplementation of 1,000 mg nicotinamide per day for 28 days reduced the number ofsubjects still suffering from at least one COVI-19 symptom at or before day 21 afterfirst testing positive for SARS-CoV-2 to a proportion of 50% (4 of 8), a surprisingly large difference compared to the silica (control) group, in which 87.5% of patients (7 of 8) still presented with one or more COVID-19 symptoms (Tables 4 and 5). One patient in the silica (control) group, but no patient in the nicotinamide group, required hospitalisation before week 2.

TABLE 4
Symptoms at week 2 (up to 21 days
after positive test for SARS-CoV-2)

| Symptoms at week 2, sorted | Total | Nicotinamide | Silica |
|---|---|---|---|
| by frequency | (n = 16) | (n = 8) | (n = 8) |
| Dyspnoea/shortness of breath | 6 | 2 | 4 |
| Cough | 5 | 2 | 3 |
| Fatigue | 4 | 2 | 2 |
| Reduced physical performance | 3 | 2 | 1 |
| Impaired sense of taste | 3 | 1 | 2 |
| Impaired sense of smell | 3 | 1 | 2 |
| Headache | 2 | 1 | 1 |
| Rhinorrhoea | 2 | 1 | 1 |
| Chest pain | 2 | 0 | 2 |
| Fever (total) | 1 | 1 | 0 |
| Fever >38° C. | 1 | 1 | 0 |
| Sore throat/pharyngalgia | 1 | 0 | 1 |
| Fever <38° C. | 1 | 1 | 0 |
| Sputum production | 1 | 1 | 0 |
| Diarrhoea | 1 | 1 | 0 |
| Arthralgia | 1 | 0 | 1 |
| Anorexia/loss of appetite | 0 | 0 | 0 |
| Myalgia | 0 | 0 | 0 |
| Vomiting | 0 | 0 | 0 |

TABLE 5
Symptom-free patients at week 2 (up to 21
days after positive test for SARS-CoV-2)

| | Total | Nicotinamide | Silica |
|---|---|---|---|
| | (n = 16) | (n = 8) | (n = 8) |
| Symptom-free | 5 | 4 | 1 |
| Persisting symptoms | 11 | 4 | 7 |

At week 4, the proportion of symptom-free patients was the same in both trial groups (5 of 8 patients; Table 6).

TABLE 6
Symptom-free patients at week 4

| | Total | Nicotinamide | Silica |
|---|---|---|---|
| | (n = 16) | (n = 8) | (n = 8) |
| Symptom-free | 10 | 5 | 5 |
| Persisting symptoms | 6 | 3 | 3 |

At week 6, only those patients with symptoms persisting at week 4 were queried, and 1 of 3 patients in the nicotinamide group had become symptom-free, but none of the remaining 2 patients in the silica (control) group (Table 7).

TABLE 7
Symptom-free patients at week 6

| | Total | Nicotinamide | Silica |
|---|---|---|---|
| | (n = 6) | (n = 3) | (n = 3) |
| Symptom-free | 1 | 1 | 0 |
| Persisting symptoms | 5 | 2 | 3 |

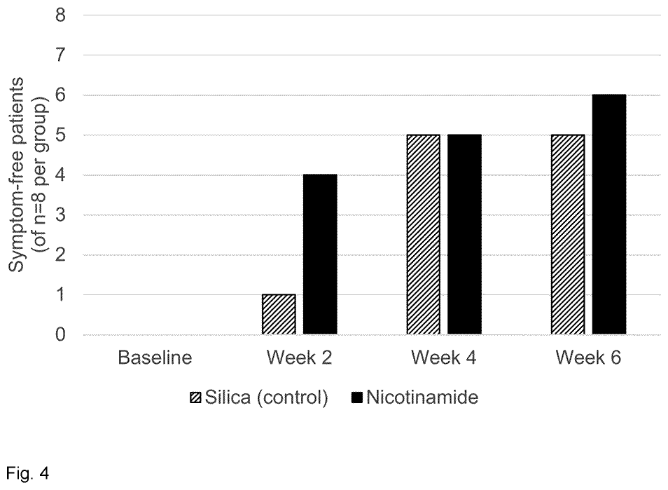

The differences in the time to resolution of symptoms are visualized in FIG. 4. The similarity of the proportion of symptom-free patients at weeks 4 and 6 indicates that the effect of nicotinamide occurs fast and early and might also indicate that the more rapid resolution of symptoms in the nicotinamide group was not due to an inadvertently unequal distribution of patients with different prognosis. It appears plausible that a considerable proportion of COVID-19 patients, who may share certain yet unknown similarities in their disease characteristics, show a much faster resolution of symptoms when supplemented with nicotinamide than others whose COVID-19 follows the typical disease course that is evident in the silica (control) group and reported in the available literature (see Background).

The beneficial effect of nicotinamide at week 2 appeared to be very strong in the recruited patient population, but was also clearly evident when the symptom-free proportion of patients was compared to results obtained with a comparable European population from France in which 68% (103 of 150) patients retained at least one symptom of their COVID-19 disease at Day 30 and, even more strikingly, 66% (86 of 130) at Day 60 (Carvalho-Schneider et al. 2021, Clin. Microbiol. Infect. 27:258). When this long-term outcome is compared to the results from week 6 in the present trial, is clear that the patient population recruited for the present trial and particularly the silica (control) group had no unusually unfavourable disease course or prognosis, but is well comparable to the published cohorts.

Taken together, nicotinamide supplementation surprisingly showed a strong benefit in reducing the time to resolution of symptoms in patients with COVID-19.

### Example 3: Formulation Variants (General Information for Performing the Invention)

(1) Nicotinamide or suitable precursors or metabolites thereof can be administered alone or in combination in a tablet with a core comprising the active substance (preferably nicotinamide) in a matrix for at least partially controlled release (e.g., comprising different grades of hydroxypropyl cellulose, hydroxypropyl methylcellulose, microcrystalline cellulose, sodium carboxymethylcellulose, starch, modified starch, pregelatinized starch, gelatin, polyvinylpyrrolidone, or combinations thereof, or employing other matrix or multi-matrix technologies). The tablet core can be provided with a coating layer system comprising an inner layer for delayed release in the lower small intestine and/or colon, a layer of active substance formulated for immediate-release and an outer layer that protects the layers below until the tablet reaches the stomach and masks the taste of the tablet. The layer for delayed release comprises a film coating which contains acrylic and/or methacrylate polymers in various mixtures for delayed release or biodegradable polymers for microbiota-dependent release. Further suitable coating agents are water-insoluble waxes, such as carnauba wax, and/or polymers, such as poly(meth)acrylates, e.g., the entire poly(meth)acrylate product portfolios with the trade names Eudraguard® and Eudragit® provided by from Evonik Industries, in particular Eudraguard® protect, Eudraguard® control, Eudraguard® biotic, Eudraguard® natural, Eudragit® L 30 D-55 (an aqueous dispersion of anionic polymers with methacrylic acid as a functional group), Eudragit® L 100-55 (which contains an anionic copolymer based on methacrylic acid and ethyl acrylate), Eudragit® L 100 or L 12,5 or S 100 or S 12,5 (anionic copolymers based on methacrylic acid and methyl methacrylate), combinations of Eudragit® S and L compounds, or Eudragit® FS 30 D (an aqueous dispersion of an anionic copolymer based on methyl acrylate, methyl methacrylate and methacrylic acid), and/or water-insoluble celluloses (e.g., methyl cellulose, ethyl cellulose). Where appropriate, water soluble polymers (e.g., polyvinylpyrrolidone), water-soluble celluloses (e.g., hydroxypropylmethyl cellulose or hydroxypropyl cellulose), emulsifiers and stabilisers (e.g., polysorbate 80), polyethylene glycol (PEG), lactose or mannitol are also contained in the coating material. The taste-masking outer layer can comprise single- or multi-layer coatings comprising, alone or in combination, e.g., hydrophobic or hydrophilic polymers (e.g., methacrylic acid and methacrylic ester copolymers like Eudragit® E, E-100, RL 300, RS 300, L30D-55 or NE 30D; Eudraguard® protect, natural or control; ethylcellulose; hydroxypropylmethylcellulose; hydroxypropylcellulose; cellulose acetate; croscarmellose; polyvinyl alcohol; polyvinylpyrrolidone (e.g., PVP-K30 or Kollicoat); polyvinyl acetate; shellac; guar gum), lipids (e.g., glyceryl palmitostearate, glyceryl monostearate or glycerol behenate), talc, detergents (e.g., sodium lauryl sulfate or polysorbates like polysorbate 80), sugars and/or sweeteners.

(2) Nicotinamide or suitable precursors or metabolites thereof can be administered in a tablet based on or analogous to the OralogiK technology (BOO Pharma), with an immediate-release, delay and later pulse release kinetic as described by the manufacturer.

(3) Nicotinamide or suitable precursors or metabolites thereof can be granulated in an immediate-release mini- or micropellet formulation, of which a fixed or variable part can be provided with a coating that effects delayed (e.g. pH-dependent) release and the other part may optionally be provided with a taste-masking coating for immediate release [for details, see Example 3 (1)]. The two types of pellets can be filled together into a single sachet, stick pack or capsule in a fixed proportion, e.g. 2:1 or 1:1 for immediate:delayed release. Alternatively, the two types of pellets can be filled into separate sachets, stick packs or capsules and are administered depending on the symptoms of the patient. In further alternatives, the pellets can be filled into capsules or incorporated into tablets.

(4) Nicotinamide or suitable precursors or metabolites thereof can be administered alone or in combination in a tablet with a core comprising the active substance (preferably nicotinamide). The core can have matrix properties for at least partially controlled release and a delayed release coating as described in Example 3 (1), but no additional layers of immediate release active substance and outer coating.

(5) Nicotinamide or suitable precursors or metabolites thereof can be administered alone or in combination in a tablet with a core comprising the active substance (preferably nicotinamide). The core can have different matrix properties than those described in Examples 3 (1) and (4), but can have a delayed release coating as described in Example 3 (4).

(6) Nicotinamide or suitable precursors or metabolites thereof can be administered alone or in combination in a tablet with a core comprising the active substance (preferably nicotinamide). The core can have matrix properties for at least partially controlled release as described in Example 3 (1) and can be equipped with taste-masking technologies as described in the Detailed Description, e.g. a taste-masking coating providing immediate release starting in the stomach as described in Example 3 (1).

(7) Nicotinamide or suitable precursors or metabolites thereof can be administered alone or in combination in a tablet with a core comprising the active substance (preferably nicotinamide). The core can have no matrix properties for controlled release and can be equipped with taste-masking technologies as described in Example 3 (6).

### Example 4: Other Viral Infections (General Information for Performing the Invention)

Nicotinamide or suitable precursors or metabolites thereof are administered for the prevention or amelioration of Persistent Somatic Symptoms (particularly fatigue) of viral infections including but not limited to SARS-CoV-1, SARS-CoV-2, middle-east respiratory syndrome coronavirus (MERS-CoV); influenza; human immunodeficiency virus; hepatitis virus type A, B, C, D or E; enterovirus; or vaccinia virus, using a conventional marketed immediate-release tablet or capsule or using a formulation variant comprising but not restricted to those described in Example 3.

### Example 5: Results from the COVit-1 Trial

The “COVit-1” trial described in Example 2 was expanded to 56 patients (n=28 each with nicotinamide or silica/control) and is referred to as “COVit-1” to differentiate it from the second, larger part of the COVit trial (“COVit-2”) that was started immediately after COVit-1 and is described in Example 6.

Table 1 shows the baseline characteristics of the COVit-1 trial population. As there was no stratification for comorbidities or other characteristics due to the rather small sample size, patients in the nicotinamide group inadvertently had significantly more comorbidities than those in the silica (control) group (p=0.014, Table 1).

TABLE 1
Baseline characteristics of participants included in the
primary efficacy population of the COVit-1 trial (n = 56)

| | Nicotinamide | | Silica | | |
|---|---|---|---|---|---|
| Characteristics | (n = 28) | | (n = 28) | | P* |
| Age, years (median, | 39.0 | (31.0-55.0) | 45.5 | (31.5-53.0) | 0.926 |
| IQR) | | | | | |
| Sex, % (n) | | | | | 0.783 |
| Female | 60.7 | (17) | 64.3 | (18) | |
| Male | 39.3 | (11) | 35.7 | (10) | |
| BMI, kg/m² | 25.9 | (22.5-27.6) | 24.7 | (21.9-28.9) | 0.694 |
| Smoking status, % (n) | | | | | 0.140 |
| Non-smoker | 53.6 | (15) | 53.6 | (15) | |
| Former smoker | 35.7 | (10) | 28.6 | (8) | |
| Smoker | 10.7 | (3) | 17.9 | (5) | |
| Ethnicity, % (n) | | | | | 0.236 |
| Caucasian | 100.0 | (28) | 89.3 | (25) | |
| Other | 0 | | 10.7 | (3) | |
| Comorbidities, % (n) | | | | | 0.014 |
| No | 25.0 | (7) | 60.7 | (17) | |
| Yes: | 75.0 | (21) | 39.3 | (11) | |
| Heart disease | 3.6 | (1) | 7.1 | (2) | 1.000 |
| Hypertension | 21.4 | (6) | 10.7 | (3) | 0.469 |
| Stroke | 0 | | 0 | | — |
| Diabetes | 0 | | 3.6 | (1) | 1.000 |
| Asthma | 14.3 | (4) | 0 | | 0.112 |
| Chronic obstructive | 0 | | 0 | | — |
| pulmonary disease | | | | | |
| Other chronic lung | 7.1 | (2) | 0 | | 0.491 |
| disease | | | | | |
| Respiratory allergy | 39.3 | (11) | 28.6 | (8) | 0.573 |
| Cancer | 7.1 | (2) | 0 | | 0.491 |
| Chronic kidney disease | 10.7 | (3) | 0 | | 0.236 |
| Chronic liver disease | 3.6 | (1) | 0 | | 1.000 |
| Organ transplant | 0 | | 0 | | — |
| Concomitant | | | | | 0.179 |
| medications | | | | | |
| No | 46.4 | (13) | 64.3 | (18) | |
| Yes | 53.6 | (15) | 35.7 | (10) | |
| Concomitant dietary | | | | | 0.108 |
| supplements | | | | | |
| No | 57.1 | (16) | 35.7 | (10) | |
| Yes | 42.9 | (12) | 64.3 | (18) | |
| Lung inflammation, | | | | | |
| % (n) | | | | | |
| No | 49.1 | (27) | 50.9 | (28) | |
| Yes | 0 | | 0 | | |
| Number of COVID-19 | 9.0 | (7.0-11.5) | 10.0 | (7.5-12.0) | 0.314 |
| symptoms (median, | | | | | |
| IQR) | | | | | |
| Emergency room | | | | | 1.000 |
| visit, % (n) | | | | | |
| No | 92.9 | (26) | 96.4 | (27) | |
| Yes - reason (n): | 7.1 | (2) | 3.6 | (1) | |

| | | |
|---|---|---|
| Shortness of breath | 1 | — |
| High fever | 1 | — |
| Cough | — | 1 |

Categorical variables are reported as count and percentage. Continuous, skewed variables are reported as median and interquartile range (IQR).
*Chi-square test or Fisher-exact test for categorical variables; Wilcoxon rank sum test for continuous variables.

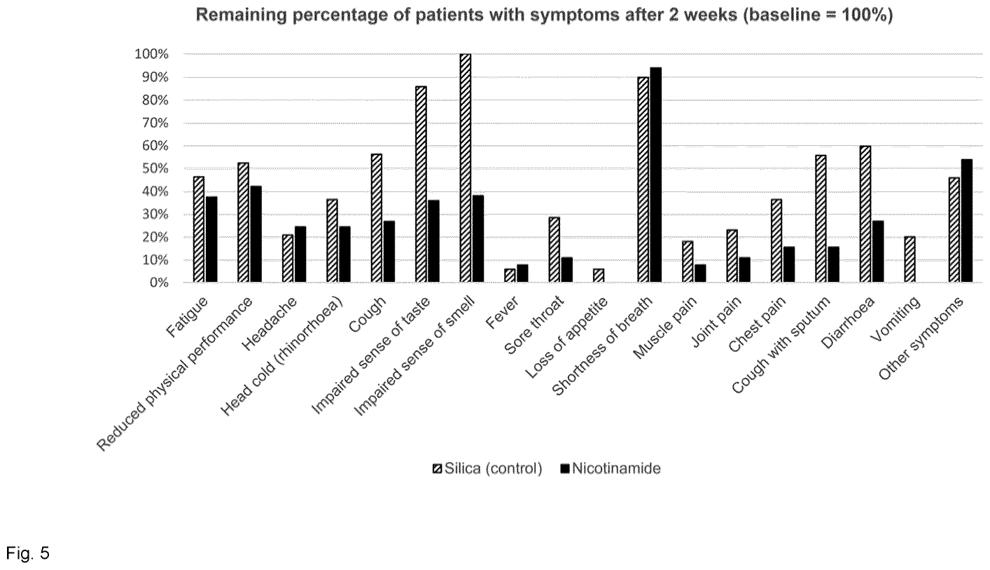

Symptom frequencies at baseline and week 2 were more strongly reduced in the nicotinamide group than in the silica (control) group, as shown in Table 2 and Table 3. For additional visualisation, FIG. 5 shows the remaining percentage of patients with symptoms (baseline=100%) for each symptom. At week 2, nicotinamide supplementation appeared to have a broad beneficial effect compared to silica (control) on diverse symptoms such as fatigue, impaired sense of smell, sore throat, cough with sputum, or joint and chest pain (Table 2, FIG. 5).

With the regard to the complete resolution of symptoms after 2 weeks of supplementation, the signal from the pilot group of n=16 patients (Example 2) was confirmed to an expectedly lesser, but still striking extent with an amplitude of approximately 20 percentage points (35.7% of patients were symptom-free after 2 weeks of nicotinamide supplementation compared to only 14.3% with placebo/silica; Table 3).

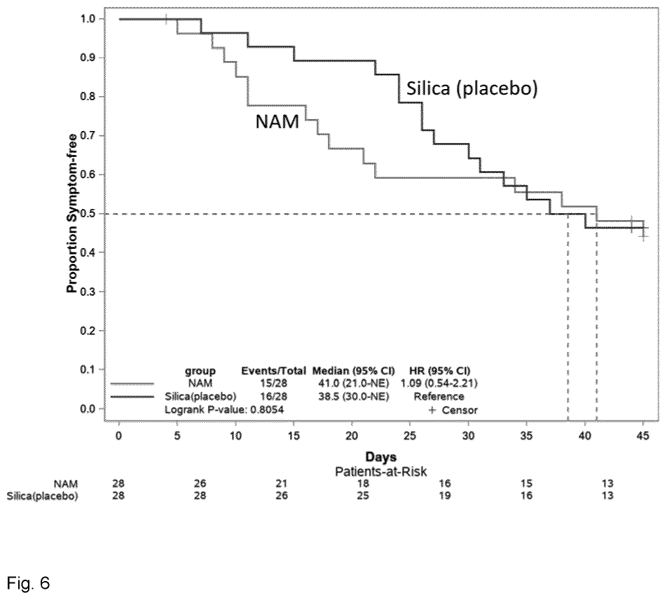

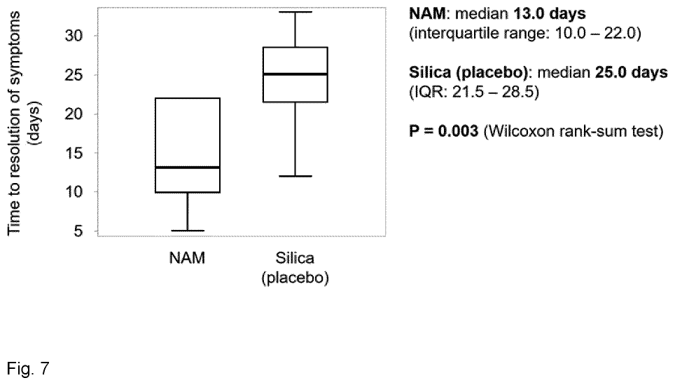

Like in the pilot group of n=16 patients, the similarity of the proportion of symptom-free patients at week 4 (Table 4) indicates that the effect of nicotinamide occurs fast and early. This is also reflected in the Kaplan-Meier curves for complete resolution of symptoms (FIG. 6). In agreement with the results described above, a highly significant difference in the median time to resolution of symptoms (in days) was detected at week 4. This period was only approximately half as long in patients supplemented with nicotinamide compared to those receiving silica (Table 4; FIG. 7). Surprisingly, female patients selectively benefited from conventional nicotinamide supplementation in the COVit-1 trial population (Table 3; Table 4).

TABLE 2
Reduction of symptoms at week 2 compared
to baseline in the COVit-1 trial (n = 56)

| COVID-19 | | | | | | |
|---|---|---|---|---|---|---|
| symptoms | Nicotinamide | | Silica (control) | | Nicotinamide | |
| (sorted by | (n = 28) | | (n = 28) | | vs. silica | |
| frequency | Base- | Week | Base- | Week | OR estimate | |
| at baseline) | line | 2 | line | 2 | (95% CI) | |
| Fatigue | 82.1% | 30.8% | 92.9% | 42.9% | 2.72 | (0.58-12.7) |
| Reduced | 82.1% | 34.6% | 82.1% | 42.9% | 1.19 | (0.49-2.90) |
| physical | | | | | | |
| performance | | | | | | |
| Headache | 78.6% | 19.2% | 85.7% | 17.9% | 1.27 | (0.35-4.53) |
| Head cold | 78.6% | 19.2% | 78.6% | 28.6% | 1.41 | (0.35-6.32) |
| (rhinorrhoea) | | | | | | |
| Cough | 71.4% | 19.2% | 57.1% | 32.1% | 1.01 | (0.11-9.00) |
| Impaired | 64.3% | 23.1% | 50.0% | 42.9% | 1.24 | (0.30-5.10) |
| sense of | | | | | | |
| taste | | | | | | |
| Impaired | 60.7% | 23.1% | 50.0% | 50.0% | 2.05 | (0.26-16.3) |
| sense of | | | | | | |
| smell | | | | | | |
| Fever | 50.0% | 3.9% | 60.7% | 3.6% | 1.40 | (0.08-23.9) |
| Sore throat | 35.7% | 3.9% | 75.0% | 21.4% | 9.25 | (1.02 (84.1) |
| Loss of | 42.9% | 0.0% | 60.7% | 3.6% | >>^{‡} | |
| appetite | | | | | | |
| Shortness of | 28.6% | 26.9% | 35.7% | 32.1% | 1.45 | (0.38-5.56) |
| breath | | | | | | |
| Muscle pain | 50.0% | 3.9% | 39.3% | 7.1% | 1.09 | (0.19-6.25) |
| Joint pain | 35.7% | 3.9% | 46.4% | 10.7% | 2.74 | (0.45-16.8) |
| Chest pain | 25.0% | 3.9% | 39.3% | 14.3% | 12.7 | (0.25-641.0) |
| Cough with | 25.0% | 3.9% | 32.1% | 17.9% | 4.64 | (0.55-39.0) |
| sputum | | | | | | |
| Diarrhoea | 28.6% | 7.7% | 17.9% | 10.7% | 0.84 | (0.18-3.93) |
| Vomiting | 3.6% | 0.0% | 17.9% | 3.6% | >>^{‡‡} | |
| Other | 28.6% | 15.4% | 32.1% | 14.3% | 1.07 | (0.21-5.48) |
| (individually | | | | | | |
| reported) | | | | | | |

Generalized linear mixed model with random effects (for repeated measures data) to assess the effectiveness of nicotinamide and silica in reducing COVID-19 symptoms after a two-week intervention.
CI, confidence interval; OR, odds ratio.
^{‡}OR very high;
^{‡‡}OR very high (unstable due to the small sample: only 1 patient in the nicotinamide group vs. 5 patients in the silica group reported vomiting at baseline which was resolved (except for 1 patient in the silica group) by week 2.

TABLE 3
Relative Risk and 95% Confidence Level of being COVID-19 symptom-free
at week 2 according to treatment groups in the COVit-1 trial (n = 56)

| | Nicotinamide | | Silica (control) | | P |
|---|---|---|---|---|---|
| COVID-19 patients (n = 56) | 28* | | 28 | | |
| Symptom-free, % (n) | 35.7 | (10) | 14.3 | (4) | |
| Absolute Risk difference, %, 95% Confidence Level | 21.4 (−0.6; 43.4) | | | | |
| Relative Risk, 95% Confidence Level | 2.50 | (0.89-7.03) | REF | | 0.083 |
| Time from diagnosis to symptom-free (days), | 11.0 | (9.0-16.0) | 10.0 | (8.0-13.0) | 0.616^{‡} |
| median (interquartile range) | | | | | |
| Female COVID-19 patients (n = 35) | 17 | | 18 | | |
| Symptom-free, % (n) | 47.1 | (8) | 11.1 | (2) | |
| Absolute Risk difference, %, 95% Confidence Level | 35.9 (8.1-63.8) | | | | |
| Relative Risk, 95% Confidence Level | 4.24 | (1.04; 17.2) | REF | | 0.043 |
| Time from diagnosis to symptom-free (days), | 10.5 | (8.5-13.5) | 8.0 | (7.0-9.0) | 0.293^{‡} |
| median (interquartile range) | | | | | |
| Male COVID-19 patients (n = 21) | 11* | | 10 | | |
| Symptom-free, % (n) | 18.2 | (2) | 20.0 | (2) | |
| Absolute Risk difference, %, 95% Confidence Level | 1.8 (−35.5; 31.9) | | | | |
| Relative Risk, 95% Confidence Level | 0.91 | (0.16; 5.30) | REF | | 0.916 |
| Time from diagnosis to symptom-free (days), | 13.0 | (11.0-15.0) | 14.0 | (11.0-17.0) | 1.000^{‡} |
| median (interquartile range) | | | | | |

*Two male patients assigned to nicotinamide missed the week 2 interview and were therefore conservatively considered as treatment failures according to an “intention to treat” approach.
^{‡}Wilcoxon rank-sum test.

TABLE 4
Relative Risk and 95% Confidence Level of being COVID-19 symptom-free
at week 4 according to treatment groups in the COVit-1 trial (n = 56)

| | Nicotinamide | | Silica (control) | | P |
|---|---|---|---|---|---|
| COVID-19 patients (n = 56) | 28* | | 28 | | |
| Symptom-free, % (n) | 39.3 | (11) | 42.9 | (12) | |
| Absolute Risk difference, %, 95% Confidence Level | −3.57 (−22.2; 29.3) | | | | |
| Relative Risk, 95% Confidence Level | 0.92 | (0.49-1.72) | REF | | 0.786 |
| Time from diagnosis to symptom-free (days), | 13.0 | (10.0-22.0) | 25.0 | (21.5-28.5) | 0.003^{‡} |
| median (interquartile range) | | | | | |
| Female COVID-19 patients (n = 35) | 17 | | 18* | | |
| Symptom-free, % (n) | 52.9 | (9) | 50.0 | (9) | |
| Absolute Risk difference, %, 95% Confidence Level | 2.94 (−30.2; 36.1) | | | | |
| Relative Risk, 95% Confidence Level | 1.06 | (0.56; 2.02) | REF | | 0.862 |
| Time from diagnosis to symptom-free (days), | 13.0 | (10.0-22.0) | 26.0 | (24.0-27.0) | 0.002^{‡} |
| median (interquartile range) | | | | | |
| Male COVID-19 patients (n = 21) | 11* | | 10 | | |
| Symptom-free, % (n) | 18.2 | (3) | 30.0 | (3) | |
| Absolute Risk difference, %, 95% Confidence Level | −0.12 (−48.2; 24.6) | | | | |
| Relative Risk, 95% Confidence Level | 0.61 | (0.13; 2.92) | REF | | 0.532 |
| Time from diagnosis to symptom-free (days), | 16.5 | (11.0-22.0) | 17.0 | (12.0-30.0) | 0.773^{‡} |
| median (interquartile range) | | | | | |

*Two male patients assigned to nicotinamide and two female patients assigned to silica missed the week 4 interview and were therefore conservatively considered as treatment failures according to an “intention to treat” approach.
^{‡}Wilcoxon rank-sum test.

Given the fact that the nicotinamide group inadvertently contained significantly more patients with comorbidities (see above), with some of these predisposing for a more severe course of COVID-19, the highly significant advantage of nicotinamide with regard to the time to resolution of symptoms (Table 4) is surprising. Even more surprising was the gender-specificity of this effect with only females benefiting from conventional nicotinamide supplementation.

### Example 6: Interim Results from the COVit-2 Trial

Background and Methods

After the positive signals from the COVit-1 trial (Examples 2 and 5), the second part of the COVit trial (COVit-2) was started and is ongoing at the time of submission of this application. The trial structure and procedures principally remained the same as described in Example 2, but with major changes regarding the trial supplements. In COVit-2, patients randomized to nicotinamide receive a combination of two different nicotinamide tablets: one conventional 500-mg immediate-release nicotinamide tablet (the same as used in COVit-1) and one novel 500-mg controlled-ileocolonic-release nicotinamide (CICR-NAM) tablet, which ensures prolonged and continuous intestinal exposure to nicotinamide. Due to the positive signals from the COVit-1 trial, the use of a real placebo (two different matching tablets) was justified instead of administrating the placebo-like dietary supplement silica. As the vaccinations against SARS-CoV-2 became available to the broader public only after several hundred patients had already been recruited into the COVit-2 trial, partly or fully vaccinated patients were excluded from participation to maintain the same inclusion population and ensure formal comparability of all patients within the trial. However, there is no reason or evidence to assume that the fundamental metabolic and microbiota-targeting effects of nicotinamide could be any different in symptomatic COVID-19 patients with or without prior vaccination.

According to the trial protocol, an interim analysis was performed after recruitment of approximately 400 patients to test for futility and adjust the sample size, if necessary. In the following, key data for n=402 patients (n=201 supplemented with the nicotinamide tablet combination and n=201 with placebo) from this analysis are summarized. For reasons of conciseness and along the lines of Example 5, the present Example 6 focuses mainly on statistically significant differences (Bonferroni-corrected for multiple-testing) between symptoms at baseline and at week 2. Moreover, recent advances in COVID-19 research and therapy (see Background) suggested to focus on patients with at least one characteristic or underlying medical condition associated with an increased risk of developing severe illness from COVID-19 (cf. the trials for molnupiravir and PAXLOVID™ described in the Background section). After blinded analysis of risk factor frequencies in the population, the following six risk groups were defined:
age of at least 60 years;
body mass index of at least 30.0 or type 2 diabetes;
cardiovascular diseases, high blood pressure or stroke;
asthma, chronic obstructive pulmonary disease or other chronic lung diseases;
current or former smokers (the latter being defined as patients who smoked more than 100 cigarettes or other smoking products in total so far, but have not smoked for at least 4 weeks);
all patients with at least one characteristic or underlying medical condition associated with an increased risk of developing severe illness from COVID-19, comprising all risk groups above and additionally all patients with type 1 diabetes, chronic kidney diseases, chronic liver diseases, cancer, organ transplants, current immunosuppressive therapy or chronic neurological diseases (multiple sclerosis, Parkinson's disease).

Moreover, seven groups of patients with up to three typical COVID-19 lead symptoms at baseline were defined, i.e. patients with cough and/or fever and/or taste loss in all combinations.

All of these indicated patient groups were analyzed for both dichotomous and ordinal variables. The dichotomous variables (yes/no) included fatigue, reduced physical performance, shortness of breath, loss of taste and cough (in this order). These variables were analyzed using Chi-Square analysis or Fisher-Exact test (in case of more than 20% of cells having expected frequencies<5). The ordinal variables (based on complaint scales with the grades 0=normal, 1=very minor complaint, 2=minor complaint, 3=moderately bad, 4=bad, 5=very bad, and 6=maximal complaint) included the ability to perform normal activities, cough and shortness of breath (in this order). These variables were analyzed using the t-test if the data group exhibited parametric distribution or with the Mann-Whitney U test if the data group exhibited non-parametric distribution. During the ordinal variable analysis, the possible effect of timepoints was considered by analysis of the differences between the later weeks (weeks 2, 4, and 6) and baseline (week 0). The numerical value of these differences allowed for further analysis by recoding the symptoms as worsening, constant, or improving from baseline. The recoded values were then analyzed using Chi-Square tests to determine the significance of these categories.

Results from Patients with an Increased Risk for Severe COVID-19

Patients with at least one characteristic or underlying medical condition associated with an increased risk of developing severe illness from COVID-19

TABLE 1.1
Changes in fatigue at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 117 | | | | | | n = 115 | | | | | | | | |
| | worse | % | constant | % | improved | % | worse | % | constant | % | improved | % | X² | df | p-value |
| W2-BL | 3 | 2.56% | 67 | 57.26% | 47 | 40.17% | 6 | 5.22% | 77 | 66.96% | 32 | 27.83% | 4.5256 | 2 | 0.1041 |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W6-BL | 2 | 1.71% | 45 | 38.46% | 70 | 59.83% | 4 | 3.48% | 49 | 42.61% | 62 | 53.91% | 1.3046 | 2 | 0.5209 |

Bold italic, statistically significant difference, also after Bonferroni correction for multiple testing (α level 0.05); W2/W4/W6-BL: week 2/4/6 compared to baseline.

TABLE 1.2
Changes in reduced physical performance at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 117 | | | | | | n = 115 | | | | | | | | |
| | worse | % | constant | % | improved | % | worse | % | constant | % | improved | % | X² | df | p-value |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W4-BL | 6 | 5.13% | 56 | 47.86% | 55 | 47.01% | 8 | 6.96% | 59 | 51.30% | 48 | 41.74% | 0.8225 | 2 | 0.6628 |
| W6-BL | 6 | 5.13% | 47 | 40.17% | 64 | 54.70% | 5 | 4.35% | 52 | 45.22% | 58 | 50.43% | 0.6213 | 2 | 0.733 |

Bold italic, statistically significant difference, also after Bonferroni correction for multiple testing (α level 0.05); W2/W4/W6-BL: week 2/4/6 compared to baseline.

TABLE 1.3
Changes in the ability to perform normal activities at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 117 | | | | | | n = 115 | | | | | | | | |
| | worse | % | constant | % | improved | % | worse | % | constant | % | improved | % | X² | df | p-value |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W4-BL | 8 | 6.84% | 10 | 8.55% | 99 | 84.62% | 8 | 6.96% | 24 | 20.87% | 83 | 72.17% | 7.1546 | 2 | 0.028 |
| W6-BL | 7 | 5.98% | 14 | 11.97% | 96 | 82.05% | 7 | 6.09% | 18 | 15.65% | 90 | 78.26% | 0.6764 | 2 | 0.7131 |

[symbol]

[symbol]

, statistically significant difference, also after Bonferroni correction for multiple testing (α level 0.05); W2/W4/W6-BL: week 2/4/6 compared to baseline.

TABLE 1.4
Changes in the ability to perform normal activities at weeks 2, 4 and 6 compared to baseline (Mann-Whitney U analysis)

| | Nicotinamide | | | Placebo | | | | | |
|---|---|---|---|---|---|---|---|---|---|
| | n = 117 | | | n = 115 | | | | | |
| | Mean | Median | Standard deviation | Mean | Median | Standard deviation | W | df | p-value |
| Baseline | 3.188 | 3.0 | 1.537 | 2.730 | 3.0 | 1.602 | 12330 | 1 | 0.0342 |
| Week 2 | 1.299 | 1.0 | 1.446 | 1.583 | 2.0 | 1.510 | 14078.5 | 1 | 0.1658 |
| Week 4 | 0.880 | 0.0 | 1.308 | 0.974 | 0.0 | 1.373 | 13649 | 1 | 0.5798 |
| Week 6 | 0.769 | 0.0 | 1.255 | 0.626 | 0.0 | 1.096 | 13109 | 1 | 0.4992 |
| [symbol] - [symbol] | [symbol] | [symbol] | [symbol] | [symbol] | −1.0 | [symbol] | [symbol] | 1 | [symbol] |
| [symbol] - [symbol] | [symbol] | [symbol] | [symbol] | [symbol] | −2.0 | [symbol] | [symbol] | [symbol] | [symbol] |
| Week 6 - baseline | −2.419 | −3.0 | 1.839 | −2.104 | −2.0 | 1.784 | 14063.5 | 1 | 0.1877 |

[symbol]

[symbol]

, statistically significant difference, also after Bonferroni correction for multiple testing (α level 0.05).

Due to the sufficiently large group sizes, the randomization algorithm's stratification for gender ensured rather equal proportions of males and females in the b nicotinamide (39.3% male, 60.7% female) and placebo groups (40.0% male, 60.0% female).

Patients with a Body Mass Index of at Least 30.0 or Type 2 Diabetes

TABLE 2.1
Changes in the ability to perform normal activities at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 27 | | | | | | n = 33 | | | | | | | | |
| | worse | % | constant | % | improved | % | worse | % | constant | % | improved | % | X² | df | p-value |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W4-BL | 3 | 11.11% | 4 | 14.81% | 20 | 74.07% | 4 | 12.12% | 11 | 33.33% | 18 | 54.55% | 2.9442 | 2 | 0.2294 |
| W6-BL | 4 | 14.81% | 3 | 11.11% | 20 | 74.07% | 4 | 12.12% | 9 | 27.27% | 20 | 60.61% | 2.4242 | 2 | 0.2976 |

[symbol]

[symbol]

, statistically significant difference, also after Bonferroni correction for multiple testing (α level 0.05); W2/W4/W6-BL: week 2/4/6 compared to baseline.

TABLE 2.2
Changes in the ability to perform normal activities at weeks 2, 4 and 6 compared to baseline (Mann-Whitney U analysis)

| | Nicotinamide | | | Placebo | | | | | |
|---|---|---|---|---|---|---|---|---|---|
| | n = 27 | | | n = 33 | | | | | |
| | Mean | Median | Standard deviation | Mean | Median | Standard deviation | W | df | p-value |
| Baseline | 3.037 | 3.000 | 1.556 | 2.455 | 3.000 | 1.603 | 922 | 1 | 0.1403 |
| Week 2 | 1.556 | 1.000 | 1.577 | 1.818 | 2.000 | 1.648 | 787.5 | 1 | 0.5855 |
| Week 4 | 1.370 | 1.000 | 1.391 | 1.606 | 1.000 | 1.600 | 794.5 | 1 | 0.6626 |
| Week 6 | 1.296 | 1.000 | 1.436 | 1.091 | 0.000 | 1.422 | 859.5 | 1 | 0.5729 |
| [symbol] - [symbol] | [symbol] | [symbol] | [symbol] | [symbol] | [symbol] | [symbol] | [symbol] | [symbol] | [symbol] |
| Week 4 - baseline | −1.667 | −2.000 | 1.981 | −0.848 | −1.000 | 1.564 | 692 | 1 | 0.0523 |
| Week 6 - baseline | −1.741 | −2.000 | 1.992 | −1.364 | −2.000 | 1.765 | 770.5 | 1 | 0.4306 |

[symbol]

[symbol]

, statistically significant difference, also after Bonferroni correction for multiple testing (α level 0.05).

TABLE 2.3
Changes in the cough complaint scale at weeks 2, 4 and 6 compared to baseline (Mann-Whitney U analysis)

| | Nicotinamide | | | Placebo | | | | | |
|---|---|---|---|---|---|---|---|---|---|
| | n = 27 | | | n = 33 | | | | | |
| | Mean | Median | Standard deviation | Mean | Median | Standard deviation | W | df | p-value |
| Baseline | 3.000 | 3.000 | 1.330 | 1.970 | 2.000 | 1.075 | 1019 | 1 | 0.0039 |
| Week 2 | 1.185 | 1.000 | 1.272 | 1.030 | 0.000 | 1.489 | 879 | 1 | 0.3826 |
| Week 4 | 0.815 | 0.000 | 1.388 | 0.939 | 0.000 | 1.321 | 788 | 1 | 0.5533 |
| Week 6 | 0.370 | 0.000 | 0.967 | 0.364 | 0.000 | 0.994 | 813 | 1 | 0.8196 |
| Week 2 - baseline | −1.815 | −2.000 | 1.594 | −0.939 | −1.000 | 1.499 | 695.5 | 1 | 0.0571 |
| [symbol] - [symbol] | [symbol] | [symbol] | [symbol] | [symbol] | [symbol] | [symbol] | [symbol] | [symbol] | [symbol] |
| [symbol] - [symbol] | [symbol] | [symbol] | [symbol] | [symbol] | [symbol] | [symbol] | [symbol] | [symbol] | [symbol] |

[symbol]

[symbol]

, statistically significant difference, also after Bonferroni correction for multiple testing (α level 0.05).

Despite the medium group sizes, the randomization algorithm's stratification for gender ensured rather equal proportions of males and females in the nicotinamide (44.4% male, 55.6% female) and placebo groups (42.4% male, 57.6% female).

Patients Who are Current or Former Smokers

TABLE 3.1
Changes in reduced physical performance at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 75 | | | | | | n = 77 | | | | | | | | |
| | worse | % | constant | % | improved | % | worse | % | constant | % | improved | % | X² | df | p-value |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W4-BL | 4 | 5.33% | 29 | 38.67% | 42 | 56.00% | 7 | 9.09% | 41 | 53.25% | 29 | 37.66% | 5.2302 | 2 | 0.0732 |
| W6-BL | 4 | 5.33% | 29 | 38.67% | 42 | 56.00% | 4 | 5.19% | 38 | 49.35% | 35 | 45.45% | 1.8193 | 2 | 0.4027 |

[symbol]

[symbol]

, statistically significant difference, also after Bonferroni correction for multiple testing (α level 0.05); W2/W4/W6-BL: week 2/4/6 compared to baseline.

TABLE 3.2
Changes in the ability to perform normal activities at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 75 | | | | | | n = 77 | | | | | | | | |
| | worse | % | constant | % | improved | % | worse | % | constant | % | improved | % | X² | df | p-value |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W4-BL | 5 | 6.67% | 7 | 9.33% | 63 | 84.00% | 6 | 7.79% | 16 | 20.78% | 55 | 71.43% | 4.1294 | 2 | 0.1269 |
| W6-BL | 4 | 5.33% | 9 | 12.00% | 62 | 82.67% | 4 | 5.19% | 14 | 18.18% | 59 | 76.62% | 1.1352 | 2 | 0.5669 |

[symbol]

[symbol]

, statistically significant difference, also after Bonferroni correction for multiple testing (α level 0.05); W2/W4/W6-BL: week 2/4/6 compared to baseline.

TABLE 3.3
Changes in the ability to perform normal activities at weeks 2, 4 and 6 compared to baseline (Mann-Whitney U analysis)

| | Nicotinamide | | | Placebo | | | | | |
|---|---|---|---|---|---|---|---|---|---|
| | n = 75 | | | n = 77 | | | | | |
| | Mean | Median | Standard deviation | Mean | Median | Standard deviation | W | df | p-value |
| Baseline | 3.373 | 4.000 | 1.583 | 2.714 | 3.000 | 1.677 | 6384.5 | 1 | 0.0164 |
| Week 2 | 1.093 | 0.000 | 1.406 | 1.584 | 2.000 | 1.576 | 5250 | 1 | 0.0592 |
| Week 4 | 0.920 | 0.000 | 1.393 | 0.961 | 0.000 | 1.312 | 5594.5 | 1 | 0.5565 |
| Week 6 | 0.787 | 0.000 | 1.308 | 0.675 | 0.000 | 1.032 | 5755.5 | 1 | 0.9392 |
| [symbol] - [symbol] | [symbol] | [symbol] | [symbol] | [symbol] | [symbol] | [symbol] | [symbol] | [symbol] | [symbol] |
| [symbol] - [symbol] | [symbol] | [symbol] | [symbol] | [symbol] | [symbol] | [symbol] | [symbol] | [symbol] | [symbol] |
| Week 6 - baseline | −2.587 | −3.000 | 1.853 | −2.039 | −2.000 | 1.788 | 5244 | 1 | 0.0671 |

[symbol]

[symbol]

, statistically significant difference, also after Bonferroni correction for multiple testing (α level 0.05).

TABLE 3.4
Changes in the presence of cough at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 75 | | | | | | n = 77 | | | | | | | | |
| | worse | % | constant | % | improved | % | worse | % | constant | % | improved | % | X² | df | p-value |
| W2-BL | 3 | 4.00% | 45 | 60.00% | 27 | 36.00% | 13 | 16.88% | 43 | 55.84% | 21 | 27.27% | 7.0204 | 2 | 0.0299 |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W6-BL | 2 | 2.67% | 41 | 54.67% | 32 | 42.67% | 3 | 3.90% | 49 | 63.64% | 25 | 32.47% | 1.7447 | 2 | 0.418 |

[symbol]

[symbol]

, statistically significant difference, also after Bonferroni correction for multiple testing (α level 0.05); W2/W4/W6-BL: week 2/4/6 compared to baseline.

Due to the sufficiently large group sizes, the randomization algorithm's stratification for gender ensured rather equal proportions of males and females in the nicotinamide (37.3% male, 62.7% female) and placebo groups (40.3% male, 59.7% female).

Results from Patients with Lead Symptoms of COVID-19

Patients with Cough

TABLE 4.1
Changes in the ability to perform normal activities at weeks 2, 4 and 6 compared to baseline (Mann-Whitney U analysis)

| | Nicotinamide | | | Placebo | | | | | |
|---|---|---|---|---|---|---|---|---|---|
| | n = 106 | | | n = 94 | | | | | |
| | Mean | Median | Standard deviation | Mean | Median | Standard deviation | W | df | p-value |
| Baseline | 2.915 | 3.0 | 1.645 | 2.894 | 3.0 | 1.669 | 9427.00 | 1 | 0.9613 |
| Week 2 | 1.226 | 1.0 | 1.389 | 1.638 | 2.0 | 1.465 | 10249.50 | 1 | 0.0423 |
| Week 4 | 0.736 | 0.0 | 1.312 | 0.936 | 0.0 | 1.310 | 10040.00 | 1 | 0.0922 |
| Week 6 | 0.660 | 0.0 | 1.226 | 0.511 | 0.0 | 1.034 | 9309.50 | 1 | 0.6680 |
| [symbol] - [symbol] | [symbol] | [symbol] | [symbol] | [symbol] | [symbol] | [symbol] | [symbol] | [symbol] | [symbol] |
| Week 4 - baseline | −2.179 | −2.0 | 1.745 | −1.957 | −2.0 | 1.703 | 9939.00 | 1 | 0.2233 |
| Week 6 - baseline | −2.255 | −2.0 | 1.762 | −2.383 | −2.0 | 1.832 | 9283.500 | 1 | 0.6859 |

[symbol]

[symbol]

, statistically significant difference, also after Bonferroni correction for multiple testing (α level 0.05).

Due to the sufficiently large group sizes, the randomization algorithm's stratification for gender ensured rather equal proportions of males and females in the c nicotinamide (34.0% male, 66.0% female) and placebo groups (35.1% male, 64.9% female).

Patients with Fever

TABLE 5.1
Changes in the ability to perform normal activities at weeks 2, 4 and 6 compared to baseline (Mann-Whitney U analysis)

| | Nicotinamide | | | Placebo | | | | | |
|---|---|---|---|---|---|---|---|---|---|
| | n = 21 | | | n = 31 | | | | | |
| | Mean | Median | Standard deviation | Mean | Median | Standard deviation | W | df | p-value |
| Baseline | 4.000 | 4.0 | 1.225 | 3.355 | 3.0 | 1.199 | 652.5 | 1 | 0.0691 |
| Week 2 | 1.381 | 1.0 | 1.564 | 1.839 | 2.0 | 1.485 | 501 | 1 | 0.2932 |
| Week 4 | 1.048 | 0.0 | 1.627 | 0.903 | 0.0 | 1.326 | 562 | 1 | 0.9155 |
| Week 6 | 0.762 | 0.0 | 1.338 | 0.581 | 0.0 | 1.205 | 572 | 1 | 0.7214 |
| [symbol] - [symbol] | [symbol] | [symbol] | [symbol] | [symbol] | [symbol] | [symbol] | [symbol] | [symbol] | [symbol] |
| Week 4 - baseline | −2.952 | −3.0 | 1.322 | −2.452 | −3.0 | 1.410 | 494 | 1 | 0.2373 |
| Week 6 - baseline | −3.238 | −4.0 | 1.411 | −2.774 | −3.0 | 1.543 | 489 | 1 | 0.2019 |

[symbol]

[symbol]

, statistically significant difference, also after Bonferroni correction for multiple testing (α level 0.05).

Due to the medium group sizes, the proportions of males and females in the nicotinamide (52.4% male, 47.6% female) and placebo groups (35.5% male, 64.5% female) differed substantially despite the randomization algorithm's stratification for gender.

Patients with Cough and Fever

TABLE 6.1
Changes in the ability to perform normal activities at weeks 2, 4 and 6 compared to baseline (Mann-Whitney U analysis)

| | Nicotinamide | | | Placebo | | | | | |
|---|---|---|---|---|---|---|---|---|---|
| | n = 15 | | | n = 17 | | | | | |
| | Mean | Median | Standard deviation | Mean | Median | Standard deviation | W | df | p-value |
| Baseline | 4.000 | 4.0 | 1.464 | 3.235 | 3.0 | 1.348 | 287.50 | 1 | 0.14 |
| Week 2 | 1.733 | 2.0 | 1.580 | 2.412 | 3.0 | 1.372 | 216.00 | 1 | 0.24 |
| Week 4 | 1.200 | 0.0 | 1.781 | 1.176 | 0.0 | 1.425 | 243.00 | 1 | 0.87 |
| Week 6 | 1.067 | 0.0 | 1.486 | 0.765 | 0.0 | 1.437 | 263.00 | 1 | 0.51 |
| [symbol] - [symbol] | [symbol] | [symbol] | [symbol] | [symbol] | [symbol] | [symbol] | [symbol] | [symbol] | [symbol] |
| Week 4 - baseline | −2.800 | −3.0 | 1.373 | −2.059 | −2.0 | 1.600 | 216.50 | 1 | 0.25 |
| Week 6 - baseline | −2.933 | −3.0 | 1.580 | −2.471 | −3.0 | 1.875 | 228.00 | 1 | 0.47 |

[symbol]

[symbol]

, statistically significant difference, also after Bonferroni correction for multiple testing (α level 0.05).

Despite the medium group sizes, the randomization algorithm's stratification for gender ensured rather equal proportions of males and females in the nicotinamide (46.7% male, 53.3% female) and placebo groups (41.2% male, 58.8% female).

Patients with Cough and Taste Loss

TABLE 7.1
Changes in the cough complaint scale at weeks 2, 4 and 6 compared to baseline (Mann-Whitney U analysis)

| | Nicotinamide | | | Placebo | | | | | |
|---|---|---|---|---|---|---|---|---|---|
| | n = 36 | | | n = 37 | | | | | |
| | Mean | Median | Standard deviation | Mean | Median | Standard deviation | W | df | p-value |
| Baseline | 2.194 | 2.0 | 1.238 | 2.486 | 2.0 | 0.961 | 1200.5 | 1 | 0.1338 |
| Week 2 | 0.917 | 0.0 | 1.204 | 0.919 | 1.0 | 1.010 | 1310.5 | 1 | 0.8028 |
| Week 4 | 0.639 | 0.0 | 1.099 | 0.270 | 0.0 | 0.652 | 1440 | 1 | 0.1276 |
| Week 6 | 0.417 | 0.0 | 0.967 | 0.489 | 0.0 | 0.569 | 1378 | 1 | 0.4386 |
| Week 2 - baseline | −1.287 | −1.0 | 1.365 | −1.568 | −2.0 | 1.191 | 1410.5 | 1 | 0.3732 |
| [symbol] - [symbol] | [symbol] | [symbol] | [symbol] | [symbol] | [symbol] | [symbol] | [symbol] | [symbol] | [symbol] |
| Week 6 - baseline | −1.778 | −2.0 | 1.416 | −2.297 | −2.0 | 1.175 | 1512.5 | 1 | 0.0430 |

[symbol]

[symbol]

, statistically significant difference, also after Bonferroni correction for multiple testing (α level 0.05).

This was the only significant difference found in favour of placebo. Given the other results, this appears to be a chance finding. Despite the medium group sizes, the randomization algorithm's stratification for gender ensured rather equal proportions of males and females in the nicotinamide (36.1% male, 63.9% female) and placebo groups (29.7% male, 70.3% female).

Patients with Fever and Taste Loss

TABLE 8.1
Changes in fatigue at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 8 | | | | | | n = 11 | | | | | | | | |
| | worse | % | constant | % | improved | % | worse | % | constant | % | improved | % | X² | df | p-value |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W4-BL | 0 | 0.00% | 1 | 12.50% | 7 | 87.50% | 1 | 9.09% | 4 | 36.36% | 6 | 54.55% | 2.4647 | 2 | 0.2916 |
| W6-BL | 0 | 0.00% | 2 | 25.00% | 6 | 75.00% | 0 | 0.00% | 3 | 27.27% | 8 | 72.73% | 0.0123 | 1 | 0.9116 |

[symbol]

[symbol]

, statistically significant difference, also after Bonferroni correction for multiple testing (α level 0.05); W2/W4/W6-BL: week 2/4/6 compared to baseline.

Due to the small group sizes, the proportions of males and females in the nicotinamide (50.0% male, 50.0% female) and placebo groups (18.2% male, 81.8% female) c differed substantially despite the randomization algorithm's stratification for gender.

Patients with Cough, Fever and Taste Loss

TABLE 9.1
Changes in fatigue at weeks 2, 4 and 6 compared to baseline (Chi-Square analysis)

| | Nicotinamide | | | | | | Placebo | | | | | | | | |
|---|---|---|---|---|---|---|---|---|---|---|---|---|---|---|---|
| | n = 6 | | | | | | n = 7 | | | | | | | | |
| | worse | % | constant | % | improved | % | worse | % | constant | % | improved | % | X² | df | p-value |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| W4-BL | 0 | 0.00% | 1 | 16.67% | 5 | 83.33% | 0 | 0.00% | 3 | 42.86% | 4 | 57.14% | 1.0403 | 1 | 0.3077 |
| W6-BL | 0 | 0.00% | 2 | 33.33% | 4 | 66.67% | 0 | 0.00% | 1 | 14.29% | 6 | 85.71% | 0.6603 | 1 | 0.4164 |

[symbol]

[symbol]

, statistically significant difference, also after Bonferroni correction for multiple testing (α level 0.05); W2/W4/W6-BL: week 2/4/6 compared to baseline.

Due to the small group sizes, the proportions of males and females in the nicotinamide (50.0% male, 50.0% female) and placebo groups (14.3% male, 85.7% female) differed substantially despite the randomization algorithm's stratification for gender.

### SUMMARY

The COVit-2 trial futility analysis revealed a biologically and statistically significant positive effect of nicotinamide on related parameters—the ability to perform normal activities, fatigue and reduced physical performance—in the particularly relevant patient population with at least one characteristic or underlying medical condition associated with an increased risk of developing severe illness from COVID-19. In the risk factor subgroups of patients with obesity and/or type 2 diabetes as well as in current and former smokers, an effect on the persistence of cough was additionally observed and the effects of nicotinamide were particularly strong. Accordingly, the composition according to the invention is preferably for use for any or all of these risk factor subgroups. However, there were also effects in other risk factor subgroups that did not yet reach statistical significance and were therefore not included in the present exemplification for reasons of conciseness, but which may well reach statistical significance after the ongoing recruitment of hundreds of additional patients. Therefore, a preferred use of the composition in further risk factor subgroups is also within the scope of the invention.

As in the COVit-1 trial part, the effects were most pronounced at week 2. In patients with lead symptoms of COVID-19, the parameters in which nicotinamide was beneficial—fatigue and the ability to perform normal activities—fully overlapped with those in patients with increased risk for severe COVID-19, which adds further credibility to the signals from the interim analysis. Also with regard to subgroups based on the presence of one or more symptoms, further effects that did not yet reach statistical significance in the futility analysis and the preferred use of the composition in such subgroups of patients are within the scope of the invention.

Surprisingly, and in contrast to the use of conventional nicotinamide only (as in COVit-1; Examples 2 and 5), the combination of immediate- and controlled-ileocolonic-release nicotinamide (i) was particularly efficacious in improving key parameters of general well-being—the ability to perform normal activities, fatigue and reduced physical performance—and (ii) was beneficial in both males and females. As one example, the improvements in the scale of the ability to perform normal activities (Table 1.4) were significant in both males (p=0.005) and females (p=0.0343). If at all, a tendency towards a stronger benefit in males was observed in COVit-2 in contrast to the purely female-driven beneficial effect in COVit-1. Taken together, the data strongly suggest that nicotinamide combination formulations that at least partially release nicotinamide in the lower small intestine and/or the colon have significant and unexpected advantages compared to conventional nicotinamide formulations.

1. A method for reducing the time to resolution of one or more symptoms related to a disease selected from the group consisting of coronavirus disease 2019 (COVID-19), severe acute respiratory syndrome (SARS), middle-east respiratory syndrome (MERS), influenza, acquired immunodeficiency syndrome (AIDS), hepatitis type A, hepatitis type B, hepatitis type C, hepatitis type D, hepatitis type E, enterovirus infection and vaccinia virus infection, the method comprising administering a composition comprising an active substance selected from nicotinamide; nicotinic acid; nicotinic acid esters; tryptophan; a tryptophan dipeptide; nicotinamide adenine dinucleotide (NAD); nicotinamide adenine dinucleotide phosphate (NADP); an intermediate in the biosynthesis of NAD or NADP selected from the group consisting of N-formylkynurenine, L-kynurenine, 3-hydroxy-L-kynurenine, 3-hydroxyanthranilate, 2-amino-3-carboxymuconate semialdehyde, quinolinate, nicotinic acid mononucleotide (beta-nicotinate D-ribonucleotide), and nicotinic acid adenine dinucleotide; nicotinamide riboside; nicotinamide mononucleotide; 1-methylnicotinamide/N-methylnicotinamide; or a combination thereof, wherein the composition is formulated to partly or completely release the active substance(s) for topical supplementation or efficacy in the lower small intestine and/or the colon.

2. The method according to claim 1, the method comprising administering the composition as a post-exposure prophylaxis to prevent the onset of symptoms and/or ameliorate symptoms related to a disease selected from the group consisting of COVID-19, SARS, MERS, influenza, AIDS, hepatitis type A, hepatitis type B, hepatitis type C, hepatitis type D, hepatitis type E, enterovirus infection and vaccinia virus infection in patients that were tested positive for the respective pathogen.

3. The method according to claim 1 wherein the composition comprises one or more active substance formulations for immediate release and/or extended release and/or sustained release delivering the active substance mainly systemically to the circulation together with one or more active substance formulations for delayed release and/or delayed-controlled release delivering the active substance mainly topically to the lower small intestine and/or colon.

4. The method according to claim 1, wherein the composition contains a combination of two formulation variants of active substance formulations in a specific ratio by weight in the range of from 1:1 to 1:1000.

5. The method according to claim 1, wherein the composition contains a combination of two or more formulation variants of active substances in the same dosage form.

6. The method according to claim 1, wherein the composition contains a variable or fixed-dose combination of two or more formulation variants of active substances in separate dosage forms.

7. The method according to claim 1, wherein the composition has an active substance content; of 1 to 5000 mg per finished dosage form.

8. The method according to claim 1, wherein part of the active substance(s) is formulated for delayed or delayed-controlled release in order to enter the circulatory system only to a low degree, so that plasma peak levels of active substance(s) following administration of the delayed or delayed-controlled release formulation are reduced by at least 20% relative to the same amount of active substance(s) administered in immediate-release formulations in the same way and under the same conditions.

9. The method according to claim 1, wherein the composition is administered once daily.

10. The method according to claim 1, wherein the composition is administered with breakfast in the morning.

11. The method according to claim 9, wherein the dose has an active substance content, of 1 to 5000 mg per finished dosage form.

12. The method according to claim 1, wherein administering the composition provides for the improvement of the ability to perform normal activities and/or the improvement of physical performance and/or the amelioration of fatigue within four weeks in patients tested positive for the disease.

13. The method according to claim 1, wherein the disease is COVID-19.

14. The method according to claim 1, wherein the method comprises administering the composition to patients with at least one characteristic or underlying medical condition associated with an increased risk of developing severe illness from the viral infection.

15. The method according to claim 1, wherein the method comprises administering the composition to patients with at least one characteristic symptom of COVID-19.

16. The method according to claim 1, wherein the composition is formulated to partly or completely release nicotinamide.

17. The method according to claim 1, wherein the composition comprises nicotinamide formulations for immediate release and/or extended release and/or sustained release delivering nicotinamide mainly systemically to the circulation together with one or more nicotinamide formulations for delayed release and/or delayed-controlled release delivering nicotinamide mainly topically to the lower small intestine and/or colon.

18. The method according to claim 1, wherein the composition contains a combination of two formulation variants of nicotinamide formulations in a specific ratio by weight in the range of from 1:3 to 1:300.

19. The method according to claim 1, wherein the composition has a nicotinamide content of 10 to 4000 mg per finished dosage form.

20. The method according to claim 1, wherein the active substance is nicotinamide, wherein the nicotinamide is formulated for delayed or delayed-controlled release in order to enter the circulatory system only to a low degree, so that plasma peak levels of nicotinamide following administration of the delayed or delayed-controlled release formulation are reduced by at least 30% relative to the same amount of nicotinamide administered in immediate-release formulations in the same way and under the same conditions.
